SEQUENCE LISTING (2 sequences)

SEQ ID NO: 1 - DNA, 30 bp, HCV
ggcggaattcctggtcatagcctccgtgaa

SEQ ID NO: 2 - DNA, 31 bp, HCV
tggggatcccgtatgatacccgctgctttga

(19) United States
(12) Patent Application Publication
(10) Pub. No.: US 2020/0352974 A1
(43) Pub. Date: Nov. 12, 2020

(54) RESPONSE-GUIDED HCV THERAPY

(71) Applicant: Emory University, Atlanta, GA (US)

(72) Inventor: Raymond F. Schinazi, Miami, FL (US)

(21) Appl. No.: 16/854,554

(22) Filed: Apr. 21, 2020

Related U.S. Application Data
(63) Continuation of application No. 15/764,794, filed on Mar. 29, 2018, now abandoned.
(60) Provisional application No. 62/244,060, filed on Oct. 20, 2015.

(51) Int. Cl. A61K 31/7072; A61K 31/4178; A61K 45/06; A61K 31/4725; A61K 31/439; A61P 31/14; A61K 31/4184; A61K 31/4709; A61K 31/519; G01N 33/569

(52) U.S. Cl. CPC A61K 31/7072; A61K 31/4178; A61K 45/06; A61K 31/4725; A61K 31/439; G01N 2800/52; A61K 31/4184; A61K 31/4709; A61K 31/519; G01N 33/56983; A61P 31/14

(57) ABSTRACT

The present disclosure relates to solid dosage forms comprising anti-HCV compounds and methods of using such dosage forms to treat or prevent HCV infection. Direct-acting antiviral agents (DAAs) have a high cure rate, and favorable tolerability in persons infected with hepatitis C virus (HCV). However, shorter courses of therapy can improve adherence, affordability, and increase DAAs accessibility. The addition of an NS3 protease inhibitor to dual NS5A-NS5B (nucleoside) inhibitors enhances antiviral efficacy, and reduces treatment duration to 3 weeks (wks) in individuals with a rapid virologic response (RVR), defined as plasma HCV RNA<500, or <1,000, IU/mL by Day 2 of treatment.

### FIELD OF THE INVENTION

The present disclosure relates to formulations comprising three or more active and specific anti-HCV compounds, for use in combination therapy, methods of treating, curing, or preventing an HCV infection using the formulations, and methods of providing response-guided HCV therapy.

### BACKGROUND OF THE INVENTION

Recent estimates place the number of people infected with the hepatitis C virus (HCV) worldwide at more than 170 million, including 3 million people in the United States. The infection rate is thought to be roughly 4 to 5 times that of the human immunodeficiency virus (HIV). While in some individuals, the natural immune response is able to overcome the virus, in the majority of cases, a chronic infection is established, leading to increased risk of developing cirrhosis of the liver and hepatocellular carcinomas.

The virus responsible for HCV infection is a positive-strand RNA virus belonging to the family Flaviviridae. The HCV genome encodes a polyprotein that during the viral lifecycle is cleaved into ten individual proteins, including both structural and non-structural proteins. The six non-structural proteins, denoted as NS2, NS3, NS4A, NS4B, NS5A, and NS5B, have been shown to be required for RNA replication. In particular, the NS5A protein appears to play a significant role in viral replication, as well as in modulation of the physiology of the host cell. Compounds which inhibit the function of the NS5A protein can be used to HCV therapy.

Historically, the standard of care for treating chronic HCV infection involved administering peg-interferon-alpha in combination with ribavirin to the patient. The course of treatment was also lengthy, typically 48 weeks, often accompanied by serious adverse side effects, including depression, flu-like symptoms, fatigue, and hemolytic anemia, and ineffective in up to 50% of patients.

HCV protease inhibitors were then approved for in combination with interferon and ribavirin. The course of therapy was still lengthy and accompanied by undesirable side effects.

One relatively new therapy involves treatment with a nucleoside phosphoramidate prodrug, Sovaldi (Sofosbuvir), which inhibits the RNA polymerase that the hepatitis C virus uses to replicate its RNA. Sovaldi is commonly used together with ribavirin or in combination with peginterferon alfa and ribavirin.

More recently, a therapy known as Harvoni (a combination of Sovaldi and Ledipasvir, an NS5A inhibitor) has been approved. It is administered as a single daily pill containing 90 mg of Ledipasvir and 400 mg of Sofosbuvir.

While these treatments are extremely successful, they involve relatively long treatment times, for example, 12 weeks for Sovaldi and 8 weeks for Harvoni. The cost of these treatments is prohibitive, and patient compliance is difficult.

Accordingly, there remains a serious unmet need in HCV treatment, for therapies that take significantly less time than 8 or 12 weeks, and with less problems associated with patient compliance. The present invention provides such therapies.

### SUMMARY OF THE INVENTION

In one embodiment, the present disclosure relates to compositions and methods for treating HCV infection using combination therapy. The combination therapy includes at least one RNA polymerase inhibitor (also known as an NS5B inhibitor), at least one NS5A inhibitor, and at least one NS3 serine protease inhibitor, non-nucleoside polymerase inhibitor, and NS4A or helicase inhibitor.

Representative therapeutic agents include Sofosbuvir, Daclatasvir, Ledipasvir, GS-5816, Simeprevir, and Asunaprevir. The methods involve administering at least three of these anti-HCV agents, and, optionally, four or more of these therapeutic anti-HCV agents.

In another embodiment, the present disclosure relates to solid dosage forms comprising three or more of these agents, in a single dosage form or in two or more dosage forms. In one aspect of this embodiment, the solid dosage forms comprise all of the compounds in a single unit dosage form.

In another aspect, the disclosure relates to solid dosage forms comprising one or more tablets, pills, or capsules including (in total),

(i) two or three of:

a) SOF at a dosage of 400-1600 mg QD;

b) LDV at a dosage of 90-180 mg QD or DCV at a dosage of 60-120 mg QD; and

c) SMV at a dosage of 150-600 mg QD or ASV at a dosage of 100-400 mg BID,

(ii) a pharmaceutically-acceptable carrier or excipient.

The pharmaceutically-acceptable carrier or excipient can include a stabilizing polymer, or combination of pharmaceutically acceptable stabilizing polymers, in an amount of at least 5% by weight of the second composition; and/or

(iii) a pharmaceutically acceptable release rate-modifying polymer, or combination of pharmaceutically acceptable release rate-modifying polymers, in an amount of at least 5% by weight of the second composition;

wherein the stabilizing polymer, or combination of stabilizing polymers, and the release rate-modifying polymer, or combination of release rate-modifying polymers, can be the same or different.

The methods for treating HCV infection in a subject in need of such treatment comprise administering at least one dosage form as described herein, at least once daily, to the subject. In one aspect of this embodiment, the methods comprise administering two or three dosage forms as described herein, once or twice daily to the subject. In another aspect, the methods comprise administering three or more dosage forms as described herein, once or twice daily, individually, to the subject.

Optionally, screening is performed at various time points on the patient to determine the likelihood of successful therapy, i.e., of achieving a sustained virologic response (“SVR”).

Preferably, patients treated according to the methods described herein do not have cirrhosis. Accordingly, the method may involve pre-screening patients for the presence or absence of cirrhosis, with those without cirrhosis advancing to treatment.

It is important to determine whether a patient has a rapid virologic response (“RVR”). Those patients that do not have an RVR may not be suitable candidates to proceed with the treatment, and can be encouraged to follow a more conventional treatment. As used herein, an RVR is defined as having a plasma HCV RNA count of less than 500-1000 IU/ml within the first two days of treatment (day 3 to 7 is also acceptable but not preferred), such as around the first 48 hours of treatment. Patients with a plasma HCV RNA count of greater than 1,000 IU/ml at this time point may not be suitable candidates to proceed with the treatment, and can be encouraged to follow a more conventional treatment.

The treatment can proceed until patients have a plasma HCV RNA count of less than 25 IU/ml. The treatment ideally takes 21 days or less to achieve this plasma HCV RNA count, which is indicative of an SVR. Patients can optionally be evaluated periodically throughout the course of treatment to determine whether they have achieved this result.

Patients can optionally also be evaluated periodically throughout the course of treatment to determine whether they have virologic failure. This may occur when patients have copy numbers greater than 25 IU/ml after having had copy numbers less than 25 IU/ml. Further, an increase in HCV RNA at two consecutive measurements of great than one log₁₀ unit above nadir at any time point during treatment is indicative of virologic failure. Such patients can be placed on conventional therapy (such as Sovaldi and peginterferon/ribavirin or Harvoni) or treated for a longer period with conventional dual treatments (e.g., Harvoni or SOF/DAC). The addition of an anti-inflammatory agents including a JAK/STAT inhibitor in combination with the antiviral combination can clear the virus more effectively and faster. Representative JAK inhibitors are described in U.S. Publication No. 20140328793, and include Ruxolitinib (Jakafi, Incyte), Baracitinib, and Tofacitinib (Pfizer).

In another aspect, the disclosure relates to kits comprising one or more of the dosage forms of the present disclosure, along with suitable instructions for taking the dosage forms according to an appropriate schedule, whether or not to take the dosage forms with food, and/or when to get their plasma HCV RNA copy numbers assessed.

The present invention will be better understood with reference to the following detailed description.

### BRIEF DESCRIPTION OF THE FIGURES

FIG. 1 is a flow chart showing the protocol used in the clinical study described in Example 1. In the chart, uRVR means ultra-rapid viral response.

FIGS. 2A-C show a fit of the multiscale model to data from the 18 subjects who were treated with DAAs combinations assuming V₀ was distributed differently between subjects who were treated for 3 weeks and subjects who were treated for more than 3 weeks. Quantifiable HCV RNA loads are shown as black dots, whereas viral loads below the limit of quantification (dashed lines) are shown as red dots. Simulations using best-fit individual parameters estimated from the population fitting of the non-linear mixed effects model are shown as solid lines.

FIG. 3 is a chart showing the decline in mean hepatitis C viral load compared with model prediction, for 26 subjects that were evaluated. Symbols show the median log 10 viral load for each treatment group, whereas the black solid line shows the mean model predicted trajectory calculated by simulating Eq. (1) 1,000 times using parameters chosen randomly from the parameter distributions derived from the mixed effect modeling. The grey band shows the intraquartile range of simulation results. The dashed horizontal line indicates the assay lower limit of quantification. There appear to be three phases in the viral decline in all three treatment groups. The trend was more apparent in treatment group SOF+DCV+SMV (group 2). No significant difference was observed in viral decline rate between the three treatment groups. Significance was tested by the Kruskal-Wallis test and corrected by Dunn's test for multiple comparisons between groups. HCV denotes hepatitis C virus, SOF=Sofosbuvir, DCV=Daclatasvir, LDV=Ledipasvir, SMV=Simeprevir, and ASV=Asunaprevir. Solid lines are mean model trajectories calculated from predicted viral load. Dashed horizontal line indicates the assay lower limit of quantification.

FIGS. 4A and 4B are charts showing the fit of a multiscale model to data from 26 subjects treated with combination DAAs (direct acting anti-viral agents).

### DETAILED DESCRIPTION

This written description uses examples to illustrate the invention and also to enable any person skilled in the art to practice the invention, including making and using any compositions and performing any related methods. The patentable scope of the invention is defined by the claims, and may include other examples that occur to those skilled in the art. Such other examples are intended to be within the scope of the claims if they have structural elements that do not differ from the literal language of the claims or if they include equivalent structural elements with insubstantial differences from the literal language of the claims.

### I. Definitions

Section headings as used in this section and the entire disclosure are not intended to be limiting.

The abbreviation “HCV” means hepatitis C virus. Based on genetic differences between HCV isolates, the hepatitis C virus species is classified into seven genotypes (1-7), with several subtypes within each genotype (represented by lower-cased letters). Subtypes are further broken down into quasispecies based on their genetic diversity. Genotypes differ by 30-35% of the nucleotide sites over the complete genome. The difference in genomic composition of subtypes of a genotype is usually 20-25%. Subtypes 1a and 1b are found worldwide and cause 60% of all cases.

Genotype is clinically important in determining potential response to therapy and the required duration of such therapy. In addition people of African descent are much less likely to clear the infection when infected with genotypes 1 or 4, and a substantial proportion of this lack of response to treatment has been traced down to a single nucleotide polymorphism (SNP) on chromosome 19 that is predictive of treatment success.

HCV genotypes 1 and 4 have been distributed endemically in overlapping areas of West and Central Africa, infecting for centuries human populations carrying the genetic polymorphism in question. This has prompted scientists to suggest that the protracted persistence of HCV genotypes 1 and 4 in people of African origin is an evolutionary adaptation over many centuries to these populations' immunogenetic responses.

Where a numeric range is recited, each intervening number within the range is explicitly contemplated with the same degree of precision. For example, for the range 6 to 9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated. In the same manner, all recited ratios also include all sub-ratios falling within the broader ratio.

The term “AUC_{ifin.}” refers to the area under the plasma concentration-time curve from the time 0 (time of dosing) to infinity (o), as calculated by the linear trapezoidal method.

The term “Cₘₐₓ” refers to the maximum observed plasma concentration over the entire sampling period.

The term “C₂₄” refers to the plasma concentration at 24 hours.

The term “subject” refers to a human subject.

The term “Tₘₐₓ” refers to the time of the maximum observed plasma concentration (Cₘₐₓ)

The abbreviation “v/v” refers to volume/volume.

The abbreviation “w/v” refers to weight/volume.

The abbreviation “w/w” refers to weight/weight.

### II. Anti-HCV Compounds

The formulations disclosed herein include three or more of the compounds shown below, in one or more dosage forms. Typically, the three or more compounds include at least one HCV RNA polymerase inhibitor (also known as an NS5B inhibitor), at least one HCV NS3 serine proteinase inhibitor, and at least one HCV NS4A or NS5A inhibitor.

While approved dosages for the individual compounds are provided herein, as the course of therapy is significantly shorter than the conventional treatments, higher dosages can be tolerated. Accordingly, in some embodiments, dosages up to 400% of the currently approved dosages can be used, for any one, two, three, or more of the compounds in the combination therapy.

The individual compounds are described below.

SOF denotes Sofosbuvir (Sovaldi, Gilead Sciences), an RNA polymerase inhibitor, which has the formula:

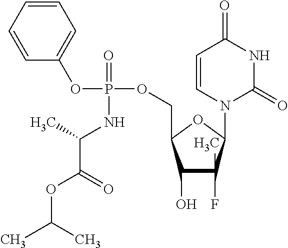

The U.S. Food and Drug Administration (US FDA) recommended dosage in adults is one 400 mg tablet, taken orally, once daily.

For patients infected with HCV of genotype 1 or 4, the current standard of care is currently Sovaldi plus peginterferon alfa plus ribavirin for 12 weeks.

For patients infected with HCV of with genotype 2, the current standard of care is Sovaldi plus ribavirin for 12 weeks.

For patients with infected with HCV of genotype 3, the current standard of care is Sovaldi plus ribavirin for 24 weeks.

LDV denotes Ledipasvir (Gilead Sciences), an NS5A inhibitor, which has the formula:

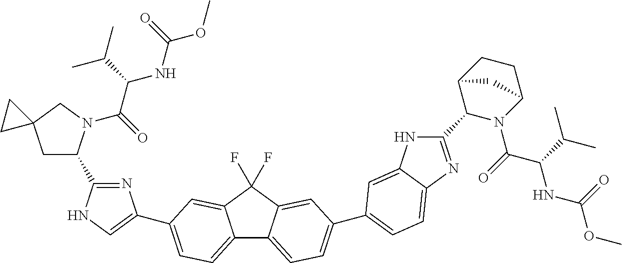 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

It is currently administered in combination with Sofosbuvir in a combination therapy known as “Harvoni,” which is a single daily pill containing 90 mg of Ledipasvir and 400 mg of Sofosbuvir.

Taken daily for 8-12 weeks, it provides cure rates of 94% to 99% in people infected with genotype 1 (the most common form of hepatitis C in the U.S. and some European countries), irrespective of the presence or absence of liver cirrhosis or prior unsuccessful treatment. It has also been evaluated for the treatment of infection with other hepatitis C genotypes, and has shown promising results in genotypes 3 and 4.

DCV denotes Daclatasvir (Daklinza, Brisol Myers Squibb), an NS5A inhibitor, which has the formula:

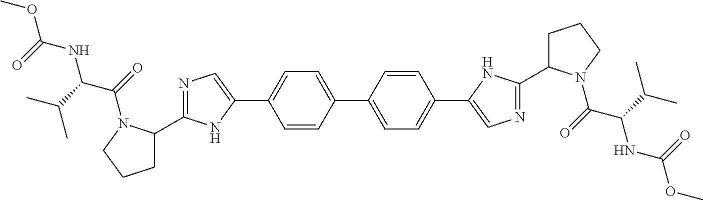

DCV is approved for Hepatitis C genotype 3 infections. It is indicated for use with Sofosbuvir, and sustained virologic response (SVR) rates are reduced in HCV genotype 3-infected patients with cirrhosis receiving DCV in combination with Sofosbuvir. The recommended dosage of DCV is 60 mg, taken orally, once daily in combination with Sofosbuvir for 12 weeks.

Additional NS5A inhibitors which can be used include Odalasvir (also known as ACH-3102, Achillion Pharmaceuticals), a second-generation NS5A inhibitor, and EDP-239 (Enanta Pharmaceuticals).

SMV denotes Simeprevir (Olysio® and Sovriad, Medivir and Jannsen Pharmaceutica) an NS3/4A protease inhibitor, which has the formula:

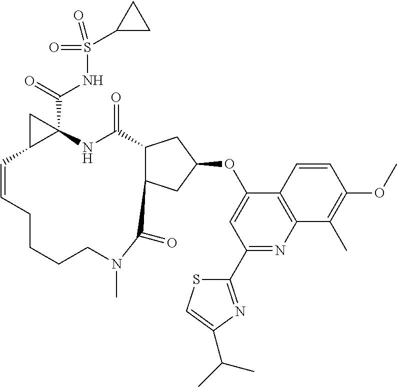 [SMILES: COc1ccc2c(O[C@@H]3C[C@H]4C(=O)N[C@]5(C(=O)NS(=O)(=O)C6CC6)C[C@H]5/C=C\CCCCN(C)C(=O)[C@@H]4C3)cc(-c3nc(C(C)C)cs3)nc2c1C]

The recommended dose of SMV is one 150-mg capsule daily, with food. It is typically taken in combination with Sofosbuvir.

ASV denotes Asunaprevir (Sunvepra, Bristol Myers Squibb), an inhibitor of the hepatitis C virus enzyme serine protease NS3, which has the formula:

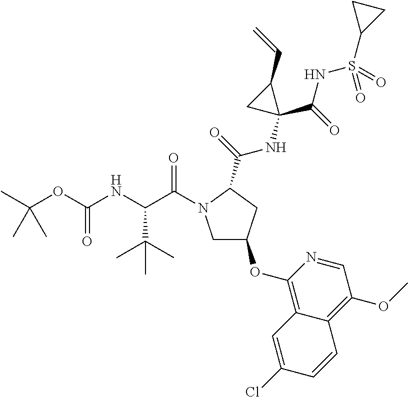 [SMILES: C=C[C@@H]1C[C@]1(NC(=O)[C@@H]1C[C@@H](Oc2ncc(OC)c3ccc(Cl)cc23)CN1C(=O)[C@@H](NC(=O)OC(C)(C)C)C(C)(C)C)C(=O)NS(=O)(=O)C1CC1]

The dosage used in clinical trials has been 200 mg Asunaprevir, twice daily (Poordad et al., May 5, 2015, Vol 313, No. 17 (May, 2015).

Sovaprevir (Achillion Pharmaceuticals), ABT-493 (Enanta Pharmaceuticals), an HCV NS3/4A protease inhibitor with broad genotype coverage, and GS-9857 (Gilead) are additional NS3 protease inhibitors that can be used.

Paritaprevir/r (a component of Viekira Pak and Technivie, Abbott Laboratories) is an acylsulfonamide inhibitor of the NS3-4A serine protease, which has the following formula:

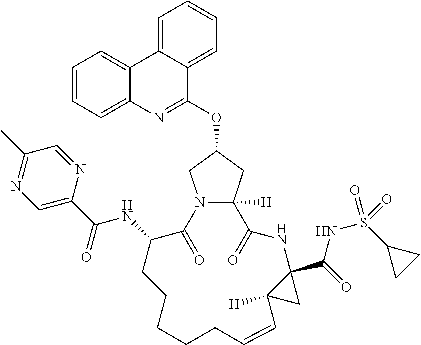 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

It has been used in the Viekira Pak in a dosage of two tablets, once daily, at a dosage of 75 mg. The US FDA recommended dosing for Paritaprevir is 150 mg/day, given as two tablets.

Ombitasvir (used in combination with paritaprevir, ritonavir and dasabuvir in the product Viekira Pak for the treatment of HCV genotype 1, and with Paritaprevir and Ritonavir in the product Technivie for the treatment of HCV genotype 4, Abbvie Laboratories), an NS5A inhibitor, which has the following formula:

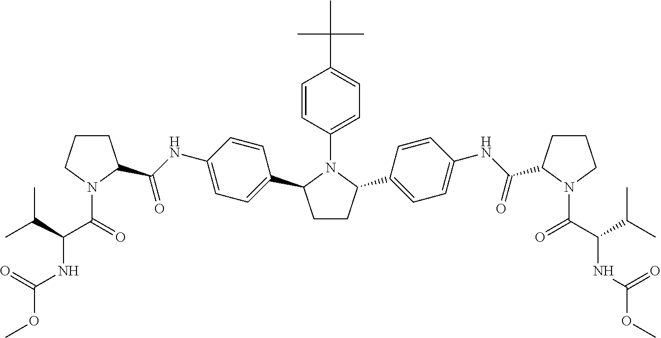

It has been used in the Viekira Pak in a dosage of two tablets, once daily, at a dosage of 12.5 mg. The US FDA recommended dosing for Ombitasvir is 25 mg/day, given as two tablets (i.e., administered twice daily).

Dasabuvir (Exviera in Europe, Abbott Laboratories, commonly used in combination with ombitasvir, paritaprevir, and ritonavir in the product Viekira Pak), is an NS5B inhibitor, with the formula:

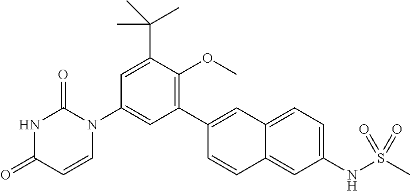 [SMILES: COc1c(-c2ccc3cc(NS(C)(=O)=O)ccc3c2)cc(-n2ccc(=O)[nH]c2=O)cc1C(C)(C)C]

It has been used in the Viekira Pak in a dosage of 250 mg, twice daily, with food. The US FDA recommended dosing for Dasabuvir is 500 mg/day, given as two tablets.

Additional NS5B inhibitors which can be used include ACH-3422, a nucleotide NS5B polymerase inhibitor (Achillion Pharmaceuticals), Beclabuvir (BMS-791325, Bristol Myers Squibb) and MK-3682 (formerly IDX21437, Merck), a uridine nucleotide analog HCV NS5B polymerase inhibitor.

Faldaprevir and Grazoprevir are currently in Phase III. Faldaprevir (Boehringer-Ingelheim) has the following formula:

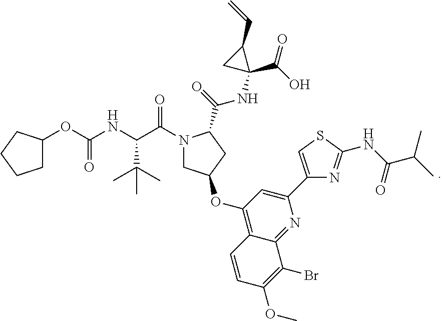 [SMILES: C=C[C@@H]1C[C@]1(NC(=O)[C@@H]1C[C@@H](Oc2cc(-c3csc(NC(=O)C(C)C)n3)nc3c(Br)c(OC)ccc23)CN1C(=O)[C@@H](NC(=O)OC1CCCC1)C(C)(C)C)C(=O)O]

Grazoprevir (Merck) has the following formula:

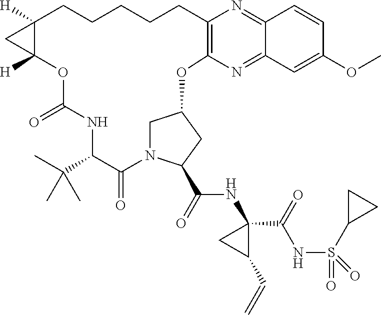 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)NS(=O)(=O)C1CC1]

Additional approved NS5A inhibitors include Elbasvir (Merck), which is in Phase III trials, and has the following formula:

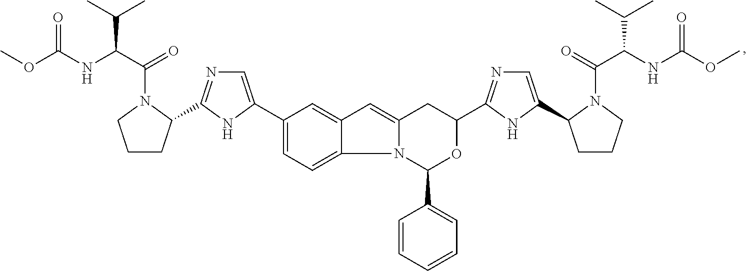

ACH-806 (Achillion Pharmaceuticals, New Haven, Conn.), which has the structure:

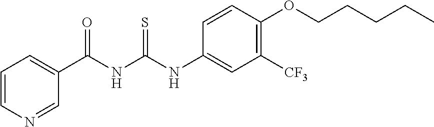 [SMILES: CCCCCOc1ccc(NC(=S)NC(=O)c2cccnc2)cc1C(F)(F)F]

Samatasvir (Merck), which is currently in Phase II trials, and has the following formula:

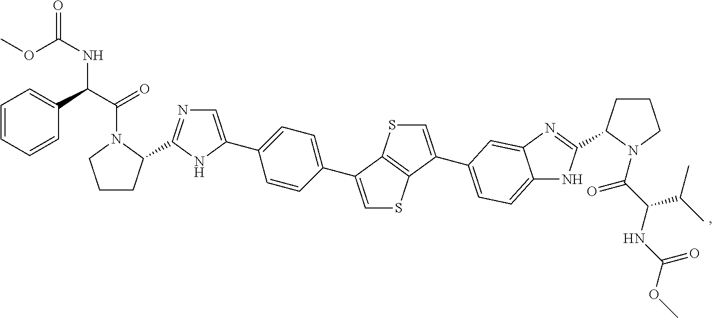 [SMILES: COC(=O)N[C@H](C(=O)N1CCC[C@H]1c1nc2cc(-c3csc4c(-c5ccc(-c6cnc([C@@H]7CCCN7C(=O)[C@H](NC(=O)OC)c7ccccc7)[nH]6)cc5)csc34)ccc2[nH]1)C(C)C]

MK-8408 (Merck), and velpatisvir (Gilead), which has the structure:

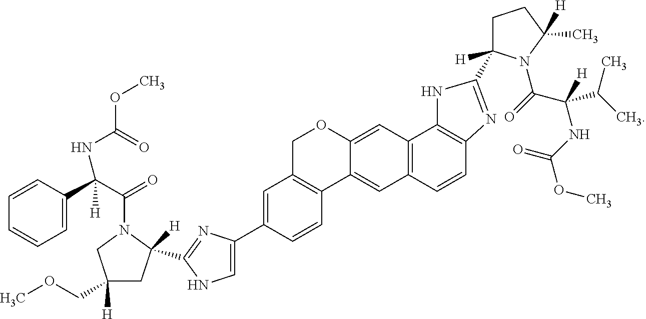 [SMILES: COC[C@H]1C[C@@H](c2nc(-c3ccc4c(c3)COc3cc5c(ccc6nc([C@@H]7CC[C@H](C)N7C(=O)[C@@H](NC(=O)OC)C(C)C)[nH]c65)cc3-4)c[nH]2)N(C(=O)[C@H](NC(=O)OC)c2ccccc2)C1]

Additional compounds include CC-31244 (Cocrystal Pharma), an non-nucleoside inhibitor (NM) of NS5B, CC-1845 (Cocrystal Pharma, a pan-genotypic NS5B inhibitor, CC-2068 and CC-2069 (Cocrystal Pharma, which are pan-genotypic, NS5A inhibitors.

Additional non-nucleoside inhibitors include TMC647055, VX-222 (VCH-222, Lomibuvir), Tegobuvir (TGV, GS-9190, Gilead Sciences), and the NNIs shown below:

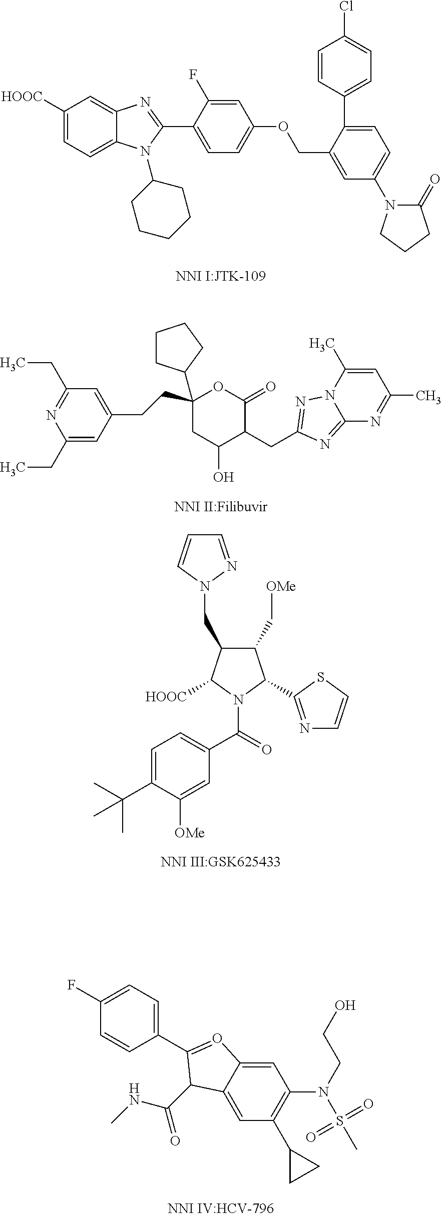

In the examples disclosed herein, the administered doses were as follows: SOF 400 mg QD; LDV 90 mg QD; DCV 60 mg QD; SMV 150 mg QD and ASV 100 mg BID, although dosages anywhere from two up to four times the administered dosages can be used, as the duration of treatment is shorter, thus minimizing side effects from larger dosages.

In one embodiment, the combination of three or more compounds includes three or more compounds selected from the group consisting of SOF (sofosbuvir), DCV (daclatasvir), LDV (ledipasvir), SMV (simeprevir), and ASV (asunaprevir).

### III. Solid Dosage Forms

The combination therapy treatment involves administration to patients in need of HCV treatment three or more of the compounds described herein, in one or more tablets.

In one embodiment, the three or more active agents are provided in two separate dosage forms, a first tablet comprising two of the three or more compounds, and a second tablet comprising the third compound, and, optionally, one or more additional compounds.

In another embodiment, the three or more active agents are provided in a single tablet.

Depending on the particular drug, and its known dosing schedules from other combination therapies, the dosages for each compound, individual, may be given once or twice a day, at varying times during the day (i.e., morning, noon, and/or night), with or without food. Initial results have indicated that the combination therapy treatment described herein is extremely effective.

Considerations for preparing multi-drug tablets include the following:

(a) Total Daily Dose: The combined daily dose of the compounds may be very large (totaling 750 mg or more), and may have to be administered in several tablets.

(b) Dosage Form Size: When administered as a single tablet, the combination therapy may have to be a relatively large dosage form (i.e., tablet, pill, capsule, and the like).

(c) Drug Loading: Drug loading limitations may impact how the combination therapy is loaded into one or more oral dosage forms.

(d) Different Pharmacokinetic Profiles: The daily dosing regimen may differ for each of the individual components, and it may be desired to deliver a specific pharmacokinetic profile for each of the three or more compounds.

(e) Solubility: Certain of the compounds used in the combination therapy described herein have low solubility. They may exhibit lower bioavailability and/or higher variability in bioavailability relative to more soluble compounds, due to their poor aqueous solubility and low dissolution.

(f) Free Acid Conversion: The free acid form of certain compounds may exhibit good permeability, but poor solubility in the gastrointestinal tract. Administering a salt of such compounds (such as the sodium salt) rather than the free acid form may or may not improve the solubility and uptake in the gastrointestinal tract.

(g) Regional Absorption: The rate and extent of which the compounds are absorbed varies throughout the gastrointestinal tract, which can make it difficult to design a once daily dosing that is bioequivalent to twice or thrice daily dosing.

Individually, the above considerations can be considered by those of skill in the art when co-formulating the three or more active ingredients in one or more suitable solid dosage forms. Collectively, these considerations can guide those of skill in the art in preparing appropriate formulations in one or more suitable solid dosage forms, without adversely impacting dosage form size and/or the number of unit dosage forms that must be administered on a daily basis while achieving suitable efficacy and bioavailability.

A. Active Ingredients

Unless otherwise stated, any reference in this disclosure to an amount of the compounds described herein is intended to refer to the free acid or free base equivalent weight of the compound. For example, 350 mg of an active compound refers to 350 mg of the free acid or free base form of the compound, or an equivalent amount of a salt (e.g., a sodium salt) of the compound.

The present disclosure relates, in part, to one or more solid dosage forms that comprise three, four, or more active ingredients, which target three or more targets on HCV (such as three or more of RNA polymerase (NS5B), NS3, NS4A, and NS5A).

B. Inactive Ingredients (Excipients)

The oral dosage forms can optionally but preferably include at least one additive selected from flow regulators, binders, lubricants, fillers, disintegrants, and plasticizers. These can be used in compressing solid dispersions to form tablets, pills, and capsules. These additives can be mixed with ground or milled solid dispersion before compacting.

Disintegrants promote a rapid disintegration of the compact in the stomach and keeps the liberated granules separate from one another. Non-limiting examples of suitable disintegrants are cross-linked polymers such as cross-linked polyvinyl pyrrolidone, cross-linked sodium carboxymethylcellulose or sodium croscarmellose.

Non-limiting examples of suitable fillers (also referred to as bulking agents) are lactose monohydrate, calcium hydrogen phosphate, microcrystalline cellulose (e.g., Avicell), silicates (such as silicium dioxide), magnesium oxide, talc, potato or corn starch, isomalt, or polyvinyl alcohol. Non-limiting examples of suitable flow regulators include highly dispersed silica (e.g., colloidal silica such as Aerosil), and animal or vegetable fats or waxes. Non-limiting examples of suitable lubricants include polyethylene glycol (e.g., having a molecular weight of from 1000 to 6000), magnesium and calcium stearates, sodium stearyl fumarate, and the like.

Various other additives or ingredients may also be used in preparing a solid composition of the present invention, for example dyes such as azo dyes, organic or inorganic pigments such as aluminum oxide or titanium dioxide, or dyes of natural origin; stabilizers such as antioxidants, light stabilizers, radical scavengers, stabilizers against microbial attack; or other active pharmaceutical ingredients.

C. Polymers for Providing Sustained Release

Polymers for providing sustained release of encapsulated agents are well known in the art. If desired, the oral dosage forms can include one or more sustained-release formulations, as such are known in the art.

When present, the amount of the release stabilizing polymer, or combination of stabilizing polymers, is generally is at least 5% by weight of the composition. Pharmaceutically-acceptable stabilizing polymers, or combinations of pharmaceutically acceptable stabilizing polymers, generally will include, for example, compressible stabilizing polymers, or compressible combinations of stabilizing polymers, and non-acidic stabilizing polymers, or non-acidic combinations of stabilizing polymers. In one aspect, the stabilizing polymer, or combination of pharmaceutically acceptable stabilizing polymers, comprises a compressible stabilizing polymer, or compressible combination of stabilizing polymers. In another aspect, the stabilizing polymer, or combination of pharmaceutically acceptable stabilizing polymers, comprises a non-acidic polymer, or non-acidic combination of stabilizing polymers.

Specific pharmaceutically acceptable stabilizing polymers, or combinations of pharmaceutically acceptable stabilizing polymers, include stabilizing polymers, or combinations of stabilizing polymers, selected from the group consisting of copovidone, polyvinylpyrrolidone, hydroxypropyl methylcellulose, polyvinyl caprolactam-polyvinyl acetate-polyethylene glycol graft copolymer (SOLUPLUS®), and combinations thereof, wherein the hydroxypropyl methylcellulose has a viscosity less than 100 centipoise in a 2% solution (i.e., a 2% aqueous solution) at a temperature of 20° C. In one aspect, the stabilizing polymer, or combination of stabilizing polymers, is selected from the group consisting of copovidone, polyvinylpyrrolidone, hydroxypropyl methylcellulose, and combinations thereof; wherein the hydroxypropyl methylcellulose has a viscosity less than 100 centipoise in a 2% solution at a temperature of 20° C. In another aspect, the stabilizing polymer, or combination of stabilizing polymers, is selected from the group consisting of homopolymers or copolymers of N-vinyl pyrrolidone and cellulose esters. In another aspect, the stabilizing polymer, or combination of stabilizing polymers, comprises copovidone. In another aspect, the stabilizing polymer, or combination of stabilizing polymers, comprises polyvinylpyrrolidone. In another aspect, the stabilizing polymer, or combination of stabilizing polymers, comprises hydroxypropyl methylcellulose having a viscosity less than 100 centipoise in a 2% solution at a temperature of 20° C. In another aspect, the stabilizing polymer, or combination of stabilizing polymers, comprises polyvinyl caprolactam-polyvinyl acetate-polyethylene glycol graft copolymer (SOLUPLUS®). In another aspect, the dosage form comprises two or more stabilizing polymers selected from the group consisting of copovidone, polyvinylpyrrolidone, hydroxypropyl methylcellulose, and polyvinyl caprolactam-polyvinyl acetate-polyethylene glycol graft copolymer (SOLUPLUS®); wherein the hydroxypropyl methylcellulose has a viscosity less than 100 centipoise in a 2% solution at a temperature of 20° C.

Release Rate-Modifying Polymers

When present, the amount of the release rate-modifying polymer, or combination of release rate-modifying polymers, is generally is at least 5% by weight of the composition. In one aspect, the amount of the release rate-modifying polymer, or combination of release rate-modifying polymers, in the composition is at least 10% by weight. In another aspect, the amount of the release rate-modifying polymer, or combination of release rate-modifying polymers, in the composition is at least 15% by weight. In another aspect, the amount of the release rate-modifying polymer, or combination of release rate-modifying polymers, in the composition is at least 20% by weight. In another aspect, the amount of the release rate-modifying polymer, or combination of release rate-modifying polymers, in the composition is 5% to 60% percent by weight. In another aspect, the amount of the release rate-modifying polymer, or combination of release rate-modifying polymers, in the composition is 10% to 50% percent by weight. In another aspect, the amount of the release rate-modifying polymer, or combination of release rate-modifying polymers, in the composition is 15% to 40% percent by weight. In another aspect, the amount of the release rate-modifying polymer, or combination of release rate-modifying polymers, in the composition is 15% to 30% percent by weight.

Pharmaceutically acceptable release rate-modifying polymers, or combinations of pharmaceutically acceptable release rate-modifying polymers, generally will include, for example, compressible release rate-modifying polymers, or compressible combinations of release rate-modifying polymers, and non-acidic release rate-modifying polymers, or non-acidic combinations of release rate-modifying polymers. In one aspect, the release rate-modifying polymer, or combination of pharmaceutically acceptable release rate-modifying polymers, comprises a compressible release rate-modifying polymer, or compressible combination of release rate-modifying polymers. In another aspect, the release rate-modifying polymer, or combination of pharmaceutically acceptable release rate-modifying polymers, comprises a non-acidic polymer, or non-acidic combination of release rate-modifying polymers.

Specific pharmaceutically acceptable release rate-modifying polymers, or combinations of pharmaceutically acceptable release rate-modifying polymers, include release rate-modifying polymers, or combinations of release rate-modifying polymers, selected from the group consisting of polyvinylpyrolidone, hydroxypropyl methylcellulose, ethylcellulose polymers, copovidone, polyvinyl acetate, methacrylate/methacrylic free acid copolymers, polyethylene glycols, polyethylene oxides, and polaxamers. In one aspect, the release rate-modifying polymers, or combinations of pharmaceutically acceptable release rate-modifying polymers, are selected from the group consisting of polyvinylpyrolidone (such as polyvinylpyrolidone (PVP) K17, PVP K25, PVP K30, and PVP K90); hydroxypropyl methylcellulose (such as hydroxypropyl methylcellulose (HPMC) E3, HPMC E5, HPMC E6, HPMC E15, HPMC E4M, HPMC E10M, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, and HPMC P550; ethylcellulose polymers (such as Ethocel 4, Ethocel 7, Ethocel 10, Ethocel 14, and Ethocel 20); copovidone (vinylpyrrolidone-vinyl acetate copolymer 60/40), polyvinyl acetate, polyvinyl caprolactam-polyvinyl acetate-polyethylene glycol graft copolymer (SOLUPLUS®), methacrylate/methacrylic free acid copolymers (such as Eudragit L100-55, Eudragit L100, and Eudragit S100); polyethylene glycols (such as polyethylene glycol (PEG) 400, PEG 600, PEG 1450, PEG 3350, PEG 4000, PEG 6000, and PEG 8000); and polaxamers (such as poloxamer 124, poloxamer 188, poloxamer 237, poloxamer 338, and poloxamer 407). In another aspect, the release rate-modifying polymer, or combination of release rate-modifying polymers, is selected from the group consisting of copovidone, polyvinylpyrrolidone, and hydroxypropyl methylcellulose. In another aspect, the release rate-modifying polymer, or combination of release rate-modifying polymers, comprises hydroxypropyl methylcellulose. In another aspect, the release rate-modifying polymer, or combination of release rate-modifying polymers, comprises hydroxypropyl methylcellulose giving an apparent viscosity at 2% weight in water at 20° C. of 80 centipoise to 120,000 centipoise at 20° C. In another aspect, the release rate-modifying polymer, or combination of release rate-modifying polymers, comprises a hydroxypropyl methylcellulose selected from the group consisting of K100, K4M, K15M, and K100M hydroxypropyl methylcelluloses.

In one embodiment of the dosage forms of the present disclosure, the stabilizing polymer comprises copovidone and the release rate-modifying polymer comprises hydroxypropyl methylcellulose.

Controlled Release Formulations

All of the U.S. patents cited in this section on controlled release formulations are incorporated by reference in their entirety.

The field of biodegradable polymers has developed rapidly since the synthesis and biodegradability of polylactic acid was reported by Kulkarni et al., in 1966 (“Polylactic acid for surgical implants,” Arch. Surg., 93:839). Examples of other polymers which have been reported as useful as a matrix material for delivery devices include polyanhydrides, polyesters such as polyglycolides and polyactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 to Langer (polyanhydrides), U.S. Pat. No. 4,767,628 to Hutchinson (polylactide, polylactide-co-glycolide acid), and U.S. Pat. No. 4,530,840 to Tice, et al. (polylactide, polyglycolide, and copolymers). See also U.S. Pat. No. 5,626,863 to Hubbell, et al which describes photopolymerizable biodegradable hydrogels as tissue contacting materials and controlled release carriers (hydrogels of polymerized and crosslinked macromers comprising hydrophilic oligomers having biodegradable monomeric or oligomeric extensions, which are end capped monomers or oligomers capable of polymerization and crosslinking); and PCT WO 9705185 filed by Focal, Inc. directed to multiblock biodegradable hydrogels for use as controlled release agents for drug delivery and tissue treatment agents.

Degradable materials of biological origin are well known, for example, crosslinked gelatin. Hyaluronic acid has been crosslinked and used as a degradable swelling polymer for biomedical applications (U.S. Pat. No. 4,957,744 to Della Valle et. al.; (1991) “Surface modification of polymeric biomaterials for reduced thrombogenicity,” Polym. Mater. Sci. Eng., 62:731 7351).

Many dispersion systems are currently in use as, or being explored for use as, carriers of substances, particularly biologically active compounds. Dispersion systems used for pharmaceutical and cosmetic formulations can be categorized as either suspensions or emulsions. Suspensions are defined as solid particles ranging in size from a few manometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, as defined in U.S. Pat. Nos. 4,622,219 and 4,725,442 issued to Haynes. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763 to Dunn, et al., discloses a method for forming an implant in situ by dissolving a nonreactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

A number of patents disclose drug delivery systems that can be used to administer the combination of the thymidine and non-thymidine nucleoside antiviral agents, or prodrugs thereof. U.S. Pat. No. 5,749,847 discloses a method for the delivery of nucleotides into organisms by electroporation. U.S. Pat. No. 5,718,921 discloses microspheres comprising polymer and drug dispersed there within. U.S. Pat. No. 5,629,009 discloses a delivery system for the controlled release of bioactive factors. U.S. Pat. No. 5,578,325 discloses nanoparticles and microparticles of non-linear hydrophilic hydrophobic multiblock copolymers. U.S. Pat. No. 5,545,409 discloses a delivery system for the controlled release of bioactive factors. U.S. Pat. No. 5,494,682 discloses ionically cross-linked polymeric microcapsules.

U.S. Pat. No. 5,728,402 to Andrx Pharmaceuticals, Inc. describes a controlled release formulation that includes an internal phase which comprises the active drug, its salt or prodrug, in admixture with a hydrogel forming agent, and an external phase which comprises a coating which resists dissolution in the stomach. U.S. Pat. Nos. 5,736,159 and 5,558,879 to Andrx Pharmaceuticals, Inc. discloses a controlled release formulation for drugs with little water solubility in which a passageway is formed in situ. U.S. Pat. No. 5,567,441 to Andrx Pharmaceuticals, Inc. discloses a once-a-day controlled release formulation. U.S. Pat. No. 5,508,040 discloses a multiparticulate pulsatile drug delivery system. U.S. Pat. No. 5,472,708 discloses a pulsatile particle based drug delivery system. U.S. Pat. No. 5,458,888 describes a controlled release tablet formulation which can be made using a blend having an internal drug containing phase and an external phase which comprises a polyethylene glycol polymer which has a weight average molecular weight of from 3,000 to 10,000. U.S. Pat. No. 5,419,917 discloses methods for the modification of the rate of release of a drug form a hydrogel which is based on the use of an effective amount of a pharmaceutically acceptable ionizable compound that is capable of providing a substantially zero-order release rate of drug from the hydrogel. U.S. Pat. No. 5,458,888 discloses a controlled release tablet formulation.

U.S. Pat. No. 5,641,745 to Elan Corporation, plc discloses a controlled release pharmaceutical formulation which comprises the active drug in a biodegradable polymer to form microspheres or nanospheres. The biodegradable polymer is suitably poly-D,L-lactide or a blend of poly-D,L-lactide and poly-D,L-lactide-co-glycolide. U.S. Pat. No. 5,616,345 to Elan Corporation plc describes a controlled absorption formulation for once a day administration that includes the active compound in association with an organic acid, and a multi-layer membrane surrounding the core and containing a major proportion of a pharmaceutically acceptable film-forming, water insoluble synthetic polymer and a minor proportion of a pharmaceutically acceptable film-forming water soluble synthetic polymer. U.S. Pat. No. 5,641,515 discloses a controlled release formulation based on biodegradable nanoparticles. U.S. Pat. No. 5,637,320 discloses a controlled absorption formulation for once a day administration. U.S. Pat. Nos. 5,580,580 and 5,540,938 are directed to formulations and their use in the treatment of neurological diseases. U.S. Pat. No. 5,533,995 is directed to a passive transdermal device with controlled drug delivery. U.S. Pat. No. 5,505,962 describes a controlled release pharmaceutical formulation.

C. Therapeutic Dose and Regimen

The dosage forms of the present invention are administered in accordance with a daily dosing regimen that orally delivers a therapeutic amount of the compounds to a subject. This daily dosing regimen generally delivers an amount of the compounds within the ranges set forth below:

SOF 400-1600 mg QD;

LDV 90-180 mg QD;

DCV 60-120 mg QD;

SMV 150-600 mg QD

ASV 100-400 mg BID.

Due to drug loading limitations and dosage form size constraints, administration of two or more of the dosage forms of the present invention typically will be required to deliver the necessary daily therapeutic dose to the subject. In one aspect, daily administration of two of the dosage forms will provide the necessary daily therapeutic dose to the subject. In another aspect, daily administration of three of the dosage forms will provide the necessary daily therapeutic dose to the subject. If desired, however, daily administration of four or more of the dosage forms can be employed to provide the necessary daily therapeutic dose to the subject.

### IV. Methods of Treatment

The combination antiviral therapy described herein is more likely to be effective if the patient a) has a low level of the hepatitis C virus in his/her blood when treatment starts, and b) has a low amount of liver damage when treatment starts.

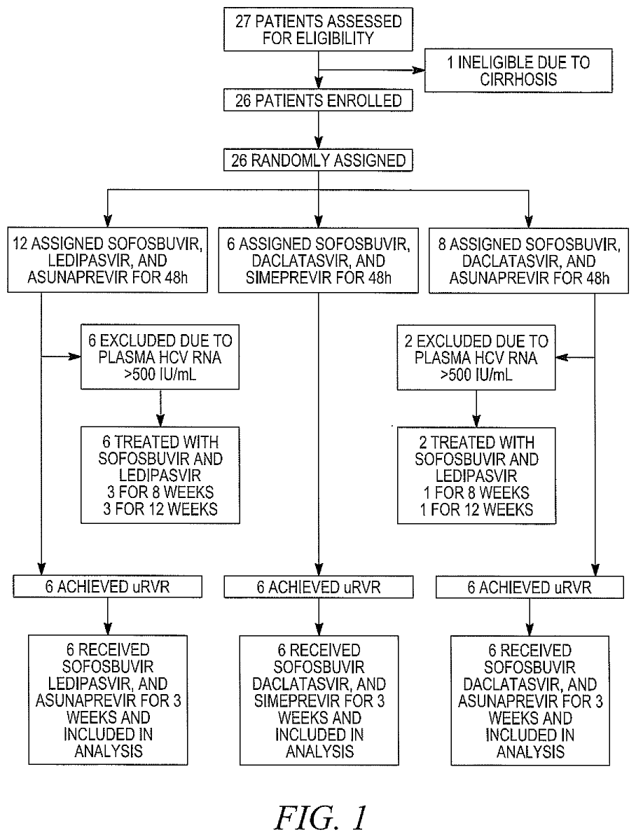

As shown in FIG. 1, a clinical trial was conducted with various combination therapies. The clinical trial is described in more detail in Example 1. However, FIG. 1 also shows one embodiment of how the treatment methods can be practiced.

Patients can optionally be evaluated for the presence of cirrhosis of the liver. Although all HCV subtypes can be treated, and patients with cirrhosis of the liver can be treated, in one embodiment, the patients are those with chronic genotype 1b (GT1b) or GT1a hepatitis C, but no cirrhosis.

Patients can be administered a combination therapy that includes an HCV RNA polymerase inhibitor (such as Sofosbuvir), an NS5A inhibitor (such as Ledipasvir or Daclastavir), and a protease inhibitor (such as Asunaprevir or Simeprevir). The other active agents described herein, which are active against the same target, can be substituted for one or more of these agents.

In one embodiment, the combination therapy includes a combination of Sofosbuvir, Ledipasvir, and Asunaprevir, at the dosages described herein.

In another embodiment, the combination therapy includes a combination of Sofosbuvir, Daclatasvir, and Simeprevir, at the dosages described herein.

In a third embodiment, the combination therapy includes Sofosbuvir, Daclatasvir, and Asunaprevir, at the dosages described herein.

In a fourth embodiment, the combination therapy includes Sofosbuvir, Daclatasvir, and Simeprevir, at the dosages described herein.

To determine which patients can be treated in a relatively short time frame, it can be important to determine which patients show a rapid virologic response (RVR). This can be done, for example, by measuring the total HCV RNA copy numbers (IU/ml), using known methodology.

At a time point approximately 36-72 hours, preferably around 42-54 hours, more preferably, around 48 hours after treatment has initiated, subjects can optionally be examined for their plasma HCV RNA copy numbers (IU/ml). This time period can optionally, but less preferably, be extended up to seven days from the start of treatment. This is an indication of whether patients have a rapid virologic response (RVR). If desired, the examination can occur sooner, but a negative result in a shorter time frame is not necessarily indicative that a patient cannot proceed with the therapy (though a positive result is indicative that a patient can proceed with the therapy).

If patients have plasma HCV RNA copy numbers higher than 1,000, and, desirably, less than 500 IU/ml, then they do not have an RVR, so are excluded from further therapy using this combination, and provided with standard therapy for a standard duration of therapy.

Patients with plasma HCV RNA copy numbers lower than 1,000, and, preferably, less than 500 IU/ml show an RVR, and can continue with the treatment. These patients can be treated for a time period of approximately three weeks, at which time plasma HCV RNA copy numbers can be measured again.

Those patients with copy numbers less than 25 IU/ml can be deemed to be “cured,” or to have sustained virologic response (SVR). Those patients with copy numbers greater than 25 IU/ml can be characterized as having “virologic failure,” and placed on conventional therapy (such as Sovaldi and peginterferon/ribavirin or Harvoni).

Optionally, but preferably, plasma HCV RNA copy numbers are measured at additional timepoints. An additional sign of virologic failure is having copy numbers greater than 25 IU/ml after having had copy numbers less than 25 IU/ml. Further, an increase in HCV RNA at two consecutive measurements of great than one log₁₀ unit above nadir at any time point during treatment is indicative of virologic failure. Such patients can be placed on conventional therapy (such as Sovaldi and peginterferon/ribavirin or Harvoni).

### VI. Methods of Preparing the Formulations

The present disclosure also relates to methods for preparing the solid dosage forms described in this specification, including those methods described in the Examples below.

In one embodiment, the disclosure relates to methods for preparing a single amorphous solid dispersion comprising one, two, or three of the compounds described herein, that can be used to in the preparation of the dosage form. The amorphous solid dispersion can be prepared by a variety of techniques such as, without limitation, melt-extrusion, spray-drying, co-precipitation, freeze drying, or other solvent evaporation techniques, with melt-extrusion and spray-drying being preferred.

In another embodiment, the method generally comprises, for example: (1) preparing a melt comprising one, two, or three of the compounds described herein, a pharmaceutically acceptable hydrophilic polymer, and a pharmaceutically acceptable surfactant; and (2) solidifying the melt. The solidified melt can comprise any amorphous solid dispersion described or contemplated herein. The method can further comprise milling the solidified melt, followed by compressing the milled product with one or more other excipients or ingredients to form a tablet core. These other excipients or ingredients can include, for example, coloring agents, flavoring agents, lubricants or preservatives.

The melt-extrusion process typically comprises the steps of preparing a melt which includes the active ingredients (i.e., one, two, or three of the compounds described herein), the hydrophilic polymer(s) and optionally the surfactant(s), and then cooling the melt until it solidifies. “Melting” means a transition into a liquid or rubbery state in which it is possible for one component to get embedded, such as homogeneously embedded, in the other component or components. In many cases, the polymer component(s) will melt and the other components including the active ingredients and surfactant(s) will dissolve in the melt thereby forming a solution. Melting usually involves heating above the softening point of the polymer(s). The preparation of the melt can take place in a variety of ways. The mixing of the components can take place before, during or after the formation of the melt. For example, the components can be mixed first and then melted or be simultaneously mixed and melted. The melt can also be homogenized in order to disperse the active ingredients efficiently. In addition, it may be convenient first to melt the polymer(s) and then to mix in and homogenize the active ingredients. In one example, all materials except surfactant(s) are blended and fed into an extruder, while the surfactant(s) is molten externally and pumped in during extrusion.

In the melt-extrusion process, the active ingredients can be employed in their solid forms, such as their respective crystalline forms. The active ingredients can also be employed as a solution or dispersion in a suitable liquid solvent such as alcohols, aliphatic hydrocarbons, esters or, in some cases, liquid carbon dioxide. The solvent can be removed, e.g. evaporated, upon preparation of the melt.

Various additives can also be included in the melt, for example, flow regulators (e.g., colloidal silica), binders, lubricants, fillers, disintegrants, plasticizers, colorants, or stabilizers (e.g., antioxidants, light stabilizers, radical scavengers, and stabilizers against microbial attack).

The melting and/or mixing can take place in an apparatus customary for this purpose such as extruders or kneaders. Suitable extruders may include single screw extruders, intermeshing screw extruders or multi-screw extruders, such as twin screw extruders, which can be co-rotating or counter-rotating and, optionally, be equipped with kneading disks. It will be appreciated that the working temperatures will be determined by the kind of extruder or the kind of configuration within the extruder that is used. Friction and shearing of the material in the extruder may provide a substantial amount of energy to the mixture and aid in the formation of a homogeneous melt of the components. However, part of the energy needed to melt, mix and dissolve the components in the extruder can be provided by heating elements.

The consistency of the melt can range from thin to pasty to viscous. Shaping of the extrudate can be conveniently carried out by a calender with two counter-rotating rollers with mutually matching depressions on their surface. The extrudate can be cooled and allowed to solidify. The extrudate can also be cut into pieces, either before solidification (hot-cut) or after solidification (cold-cut).

The solidified extrusion product can be further milled, ground or otherwise reduced to granules. The solidified extrudate, as well as each granule produced, comprises a solid dispersion, such as a solid solution, of the active ingredients in a matrix comprised of the hydrophilic polymer(s) and the pharmaceutically acceptable surfactant(s). The extrusion product can also be blended with other active ingredient(s) and/or additive(s) before being milled or ground into granules. The granules can be further processed into suitable solid oral dosage forms.

Alternatively, an amorphous solid dispersion can be prepared using the approach of solvent evaporation, via spray-drying, which provides the advantage of allowing for processing at lower temperatures, if needed, and also allows for other modifications to the process in order to further improve powder properties. The spray-dried powder can then be formulated further, if needed, and final drug product is flexible with regards to whether capsule, tablet or any other solid dosage form is desired.

Exemplary spray-drying processes and spray-drying equipment are described in K. Masters, SPRAY DRYING HANDBOOK (Halstead Press, New York, 4.sup.th ed., 1985). Non-limiting examples of spray-drying devices that are suitable for the present invention include spray dryers manufactured by Niro Inc. or GEA Process Engineering Inc., Buchi Labortechnik AG, and Spray Drying Systems, Inc. A spray-drying process generally involves breaking up a liquid mixture into small droplets and rapidly removing solvent from the droplets in a container (spray drying apparatus) where there is a strong driving force for evaporation of solvent from the droplets. Atomization techniques include, for example, two-fluid or pressure nozzles, or rotary atomizers. The strong driving force for solvent evaporation can be provided, for example, by maintaining the partial pressure of solvent in the spray drying apparatus well below the vapor pressure of the solvent at the temperatures of the drying droplets. This may be accomplished by either (1) maintaining the pressure in the spray drying apparatus at a partial vacuum; (2) mixing the liquid droplets with a warm drying gas (e.g., heated nitrogen); or (3) both.

The temperature and flow rate of the drying gas, as well as the spray dryer design, can be selected so that the droplets are dry enough by the time they reach the wall of the apparatus to be essentially solid and to form a fine powder to avoid sticking to the apparatus wall. The spray-dried product can be collected by removing the material manually, pneumatically, mechanically or by other suitable means. The actual length of time to achieve the desired level of dryness depends on the size of the droplets, the formulation, and spray dryer operation. Following the solidification, the solid powder may stay in the spray drying chamber for additional time (e.g., 5 seconds to 60 seconds) to further evaporate solvent from the solid powder. The final solvent content in the solid dispersion as it exits the dryer is generally at a sufficiently low level so as to improve the stability of the final product. For instance, the residual solvent content of the spray-dried powder can be less than 2% by weight. The residual solvent content may be within the limits set forth in the International Conference on Harmonization (ICH) Guidelines. In addition, it may be useful to subject the spray-dried composition to further drying to lower the residual solvent to even lower levels. Methods to further lower solvent levels include, but are not limited to, fluid bed drying, infra-red drying, tumble drying, vacuum drying, and combinations of these and other suitable processes.

Like the solid extrudate described above, the spray dried product contains a solid dispersion, such as a solid solution, of the active ingredients in a matrix comprised of the hydrophilic polymer(s) and the pharmaceutically acceptable surfactant(s).

Before feeding into a spray dryer, the active ingredients, the hydrophilic polymer(s), as well as other excipients such as the pharmaceutically acceptable surfactant(s), can be dissolved in a solvent. Suitable solvents include, but are not limited to, alkanols (e.g., methanol, ethanol, 1-propanol, 2-propanol or mixtures thereof), acetone, acetone/water, alkanol/water mixtures (e.g., ethanol/water mixtures), or combinations thereof. The solution can also be preheated before being fed into the spray dryer.

The solid dispersion produced by melt-extrusion, spray-drying or other techniques can be prepared into any suitable solid oral dosage forms. In one embodiment, the solid dispersion prepared by melt-extrusion, spray-drying or other techniques (e.g., the extrudate or the spray-dried powder) can be compressed into tablets. The solid dispersion can be either directly compressed, or milled or ground into granules or powders before compression. Compression can be performed in a tablet press, such as in a steel die between two moving punches.

At least one additive selected from flow regulators, binders, lubricants, fillers, disintegrants, or plasticizers may be used in compressing the solid dispersion. These additives can be mixed with ground or milled solid dispersion before compacting. Disintegrants promote a rapid disintegration of the compact in the stomach and keeps the liberated granules separate from one another. Non-limiting examples of suitable disintegrants are cross-linked polymers such as cross-linked polyvinyl pyrrolidone, cross-linked sodium carboxymethylcellulose or sodium croscarmellose. Non-limiting examples of suitable fillers (also referred to as bulking agents) are lactose monohydrate, calcium hydrogenphosphate, microcrystalline cellulose (e.g., Avicell), silicates (such as silicium dioxide), magnesium oxide, talc, potato or corn starch, isomalt, or polyvinyl alcohol. Non-limiting examples of suitable flow regulators include highly dispersed silica (e.g., colloidal silica such as Aerosil), and animal or vegetable fats or waxes. Non-limiting examples of suitable lubricants include polyethylene glycol (e.g., having a molecular weight of from 1000 to 6000), magnesium and calcium stearates, sodium stearyl fumarate, and the like.

Various other additives or ingredients may also be used in preparing a solid composition of the present invention, for example dyes such as azo dyes, organic or inorganic pigments such as aluminium oxide or titanium dioxide, or dyes of natural origin; stabilizers such as antioxidants, light stabilizers, radical scavengers, stabilizers against microbial attack; or other active pharmaceutical ingredients.

The present invention will be better understood with reference to the following non-limiting examples.

### EXAMPLES

### Example 1: Efficacy and Safety of 3-Week Response-Guided Triple Direct-Acting Antiviral Therapy for Chronic Hepatitis C Infection: a Phase 2, Open-Label, Proof-of-Concept Study

Summary:

DAAs have a high cure rate and favorable tolerability in persons infected with hepatitis C virus (HCV). However, shorter courses could improve adherence, affordability and increase DAAs accessibility. We postulated that adding an NS3 protease inhibitor to dual NS5A-NS5B (nucleoside) inhibitors would enhance antiviral efficacy and reduce treatment duration to 3 weeks (wks) in individuals with a rapid virologic response (RVR), defined as plasma HCV RNA<500 IU/mL by day 2. Accordingly, the purpose of the study was to examine the antiviral efficacy and safety of 3 weeks of response-guided therapy with an NS3 protease inhibitor and dual NS5A inhibitor-NS5B nucleotide analogue, so as to shorten the course of direct-acting antiviral agents for chronic hepatitis C virus (HCV) infection.

In this pilot, response guided therapy (RGT), open-label Phase IIa, single-center study, 26 non-cirrhotic Chinese subjects with chronic hepatitis C GT 1b [median age=34 yr (21-66), male=6, median BMI=21.7 (15.9-32.1), baseline mean HCV RNA log 10 IU/ml=6.55 (4.09-7.34)] were randomized, using a computer program, to receive at the approved doses either: sofosbuvir, ledipasvir and asunaprevir (group 1, n=12), sofosbuvir, daclatasvir and simeprevir (group 2, n=6) or sofosbuvir, daclatasvir and asunaprevir (group 3, n=8) until six patients in each group (1:1:1) achieved an ultrarapid virological response (plasma HCV RNA <500 IU/mL by day 2, measured by COBAS TaqMan HCV test, version 2.0).

Patients with an ultrarapid virological response received 3 weeks of therapy. Patients who did not achieve an ultrarapid response were switched to sofosbuvir and ledipasvir for either 8 weeks or 12 weeks. The primary endpoint was the proportion of patients with a sustained virological response at 12 weeks (SVR12) after treatment completion, analysed in the intention-to-treat population. All patients who achieved an ultrarapid virological response were included in the safety analysis. This trial is registered with ClinicalTrials.gov, number NCT02470858.

Results:

RVR was achieved in 18 (66.7%) subjects (6/12, 6/6, 6/8 for group 1, 2, 3 respectively, p=0.06). All patients with an ultrarapid virological response who were given three weeks of triple therapy achieved SVR12.

Baseline viral load was lower in subjects with RVR as compared to those without RVR (log 10 IU/mL 5.96 vs. 7.00, p<0.0001). The median time to achieve plasma HCV RNA <25 IU/mL (limit of detection) was shorter in group 1 as compared to group 3 (p=0.01). All 18 subjects who had RVR and 3 weeks DAAs achieved SVR12.

There were no discontinuations or significant adverse events reported. The most common adverse events were fatigue (one [17%] of six patients receiving sofosbuvir, ledipasvir, and asunaprevir; one [17%] of six patients receiving sofosbuvir, daclatasvir, and simeprevir; and two [33%] of six patients receiving sofosbuvir, daclatasvir, and asunaprevir) and headache (one [17%] patient in each group). No patients experienced any serious adverse events.

Conclusions: This proof-of-concept SODAPI study (ClinicalTrials.gov number NCT02470858) explored RGT to shorten the duration of HCV treatment. The results strongly suggest that administration of potent triple regimens containing NS3, NS5A and NS5B HCV-inhibitors leads to RVR (plasma HCV RNA <500 IU/mL) within 2 days in two-thirds of non-cirrhotic HCV GT 1b-infected subjects. 100% of subjects with RVR and had treatment for 3 wks, achieved SVR12, with excellent adherence and tolerability.

Thus, all patients with chronic HCV, without cirrhosis, who achieved an ultrarapid virological response on triple direct-acting antiviral regimens by day 2 and received 3 weeks of treatment were cured, with excellent adherence and tolerability. By shortening the duration of therapy from the currently recommended 12 weeks to 3 weeks, it is possible to drastically reduce the cost of therapy and the rate of adverse events.

Background

In persons with chronic hepatitis C virus (HCV) infection, shorter courses of direct-acting antiviral agents (DAAs) could improve adherence and affordability. We postulated that the addition of an NS3 protease inhibitor to dual NS5A-NS5B nucleoside analogs would enhance antiviral efficacy and shorten the treatment duration in a response guided therapy clinical study.

Introduction

There is an increasing prevalence of hepatitis C virus (HCV) worldwide and the number of people infected has increased from 2.3% to 2.8% (>122 million to >185 million people) between 1990 and 2005 (AASLD-IDSA HCV Guidance Panel. Hepatitis C guidance: AASLD-IDSA recommendations for testing, managing, and treating adults infected with hepatitis C virus. Hepatology 2015; 62: 932-54).

The estimated number of HCV infected people in China, Hong Kong, Taiwan and Korea is approximately 50 million (European Association for the Study of the Liver. EASL recommendations on treatment of hepatitis C 2015. J Hepatol 2015; 63: 199-236; Afdhal et al. Ledipasvir and sofosbuvir for untreated HCV genotype 1 infection. N Engl J Med 2014; 370: 1889-98) and the most prevalent genotype is 1b (Afdhal et al. Ledipasvir and sofosbuvir for previously treated HCV genotype 1 infection. N Engl J Med 2014; 370: 1483-93). Up to one-fifth of these persons will develop cirrhosis and a quarter of them will progress to end-stage liver disease or hepatocellular carcinoma (Kowdley et al. Ledipasvir and sofosbuvir for 8 or 12 weeks for chronic HCV without cirrhosis. N Engl J Med 2014; 370: 1879-88; Sulkowski et al., Daclatasvir plus sofosbuvir for previously treated or untreated chronic HCV infection. N Engl J Med 2014; 370: 211-21).

Treatment of hepatitis C virus (HCV) infection has entered a new era with the emergence of direct-acting antiviral agents. DAAs are well tolerated, safe, orally deliverable, and can cure almost all HCV patients within 8 to 24 weeks of treatment. By 2015, the US Food and Drug Administration (FDA) and EU had approved three new direct-acting antiviral drugs—sofosbuvir, simeprevir, and daclatasvir—for the treatment of HCV infection as part of combination regimens (AASLD-IDSA HCV Guidance Panel. Hepatitis C guidance: AASLD-IDSA recommendations for testing, managing, and treating adults infected with hepatitis C virus. Hepatology 2015; 62: 932-54; European Association for the Study of the Liver. EASL recommendations on treatment of hepatitis C 2015. J Hepatol 2015; 63: 199-236).

91-100% of individuals infected with HCV genotype 1 treated with 8-12 weeks of sofosbuvir and ledipasvir once daily (Afdhal et al. Ledipasvir and sofosbuvir for untreated HCV genotype 1 infection. N Engl J Med 2014; 370: 1889-98; Afdhal et al., Ledipasvir and sofosbuvir for previously treated HCV genotype 1 infection. N Engl J Med 2014; 370: 1483-93; Kowdley et al. Ledipasvir and sofosbuvir for 8 or 12 weeks for chronic HCV without cirrhosis. N Engl J Med 2014; 370: 1879-88).

98-100% of patients who received 12-24 weeks of sofosbuvir and daclatasvir once daily, achieved sustained virological response at 12 weeks (SVR12) (Sulkowski et al. Daclatasvir plus sofosbuvir for previously treated or untreated chronic HCV infection. N Engl J Med 2014; 370: 211-21).

The cost of such regimens is onerous, and this has adversely affected treatment access and drug compliance, and has encouraged drug counterfeiting (McCarthy M. Fake medicines are undermining global efforts to combat infectious disease, says US journal. BMJ 2015; 350: h2137; Attaran A. Stopping murder by medicine: introducing the Model Law on Medicine Crime. Am J Trop Med Hyg 2015; 92 (6 suppl): 127-32).

A major challenge is to reduce treatment cost without affecting efficacy by shortening the duration of treatment (Hagan, L M, Sulkowski M S, Schinazi R F. Cost analysis of sofosbuvir/ribavirin versus sofosbuvir/simeprevir for genotype 1 hepatitis C virus in interferon-ineligible/intolerant individuals. Hepatology 2014; 60: 37-45; Hill et al., Minimum costs for producing hepatitis C direct-acting antivirals for use in large-scale treatment access programs in developing countries. Clin Infect Dis 2014; 58: 928-36).

Attempts to reduce the duration of therapy to 6 weeks through the addition of ribavirin to sofosbuvir and ledipasvir resulted in many patients relapsing after treatment (Gane et al., Efficacy of nucleotide polymerase inhibitor sofosbuvir plus the NS5A inhibitor ledipasvir or the NS5B non-nucleoside inhibitor GS-9669 against HCV genotype 1 infection. Gastroenterology 2014; 146: 736-43). The addition of an experimental NS3/4A protease inhibitor (GS-9451) or an experimental non-nucleoside polymerase inhibitor (GS-9669) yielded an SVR12 in 95% of patients (Kohli et al., Virological response after 6 week triple-drug regimens for hepatitis C: a proof-of-concept phase 2A cohort study. Lancet 2015; 385: 1107-13).

Although the study is relatively small, it shows high rates of sustained viral response at 12 weeks using a 3-week triple direct-acting antiviral therapy, which provides in vivo support for the invention described herein, namely, ultra-short therapeutic regimens which can be effective in certain patients infected with HCV.

The study demonstrated that the duration of therapy of pan-oral direct-acting antiviral agents can be drastically shortened from the current recommended 12 weeks to only 3 weeks with triple direct-acting antiviral therapy, containing NS5B, NS3, and NS5A inhibitors, in non-cirrhotic Chinese patients infected with chronic HCV genotype 1b and who had an ultrarapid virological response. This has significant therapeutic, public health, and economic implications, because genotype 1b rather than 1a is the predominant strain in Asian populations (estimated at around 50 million infected individuals). While not wishing to be bound to a particular theory, it is believed that this therapeutic approach will also work with different HCV genotypes, and in cirrhotic patients.

Mathematical modelling of HCV RNA changes during therapy suggests that a more rapid second-phase viral decline should allow for a shorter treatment duration (Perelson A S, Guedj J. Modelling hepatitis C therapy—predicting effects of treatment. Nat Rev Gastroenterol Hepatol 2015; 12: 437-45). Nucleoside analogue inhibitors do not generate fast second-phase declines such as those with HCV protease inhibitors (Guedj et al., Hepatitis C viral kinetics with the nucleoside polymerase inhibitor mericitabine (RG7128). Hepatolgy 2012; 55: 1030-37; Guedj et al. Analysis of hepatitis C viral kinetics during administration of two nucleotide analogues: sofosbuvir (GS-7977) and GS-0938. Antivir Ther 2014; 19: 211-20; and Guedj and Perelson. Second-phase hepatitis C virus RNA decline during telaprevir-based therapy increases with drug effectiveness: implications for treatment duration. Hepatology 2011; 53: 1801-08).

Accordingly, we postulated that addition of an approved protease inhibitor, such as simeprevir or asunaprevir to sofosbuvir and ledipasvir or daclatasvir might induce a more rapid second-phase HCV RNA decline, allowing for a shorter treatment duration. Therefore, we did a proof-of-concept, response-guided therapy clinical study to investigate the efficacy and the safety of 3 weeks of triple direct-acting antiviral therapy, containing NS5B, NS3, and NS5A inhibitors, in Chinese patients with chronic HCV genotype 1b infection without cirrhosis who achieved an ultrarapid initial viral response (uRVR), defined by a serum HCV RNA lower than 500 IU/mL within the first 2 days of dosing. We focused on patients infected with genotype 1b because this is the predominant strain in Asian populations (estimated at about 50 million), although this genotype can also be found in other populations (Messina, et al. Global distribution and prevalence of hepatitis C virus genotypes. Hepatology 2015; 61: 77-87; Liu and Kao. Nanomedicines in the treatment of hepatitis C virus infection in Asian patients: optimizing use of peginterferon alfa. Int J Nanomed 2014; 9: 2051-67).

Methods

Study Design and Participants

This was an open-label, proof-of-concept, phase 2a study done at a single centre (Humanity and Health Medical Centre, Hong Kong SAR, China). Participants were identified using the Beijing 302 Hospital of PLA-Hong Kong Humanity and Health Medical Group, Hepatitis C Diagnosis and Treatment Centre database, which had records for 503 patients infected with HCV by the time of the study. People who satisfied all the inclusion and exclusion criteria and who consented were consecutively enrolled. Key inclusion criteria were: older than 18 years; documented chronic HCV genotype 1b infection for more than 6 months; a baseline plasma HCV RNA concentration of 10′-10⁷ IU/mL; and absence of cirrhosis as assessed by liver biopsy or by liver stiffness measurement less than 12.5 kPa. Key exclusion criteria were: hepatitis B virus (HBV) or HIV infection; chronic liver disease of a non-HCV aetiology; hepatocellular carcinoma or other malignancy; drug or alcohol misuse; pregnant or nursing woman; known hypersensitivity to pharmaceutical products used in this study; and any other medical disorders or clinical conditions (eg, substantial cardiopulmonary, neurological, renal, haematological, autoimmune disorders, and any malignancy) that could interfere with the study. Treatment-experienced patients had been exposed to interferon-based therapy previously.

Written informed consent was obtained from all patients.

Randomization and Masking

All eligible patients were randomly allocated by a computerized system to one of three treatment groups (sofosbuvir, ledipasvir, and asunaprevir; sofosbuvir, daclatasvir, and simeprevir; or sofosbuvir, daclatasvir, and asunaprevir) until six patients achieved a uRVR in each group. The computer sequence was generated by a biostatistician (JC) who assigned them to trial groups but was not involved in the rest of the trial.

The trial was open label; patients and investigators were aware of group assignment. This open-label, phase 2a study included an exploratory cohort of 26 non-cirrhotic Chinese subjects with chronic HCV genotype 1b infection. We randomly assigned subjects to receive sofosbuvir, ledipasvir and asunaprevir (group 1; N=12), sofosbuvir, daclatasvir and simeprevir (group 2; N=6) or sofosbuvir, daclatasvir and asunaprevir (group 3; N=8).

Patients received sofosbuvir, ledipasvir, and asunaprevir; sofosbuvir, daclatasvir, and simeprevir; or sofosbuvir, daclatasvir, and asunaprevir. Doses were as follows: sofosbuvir 400 mg once daily; ledipasvir 90 mg once daily; daclatasvir 60 mg once daily; simeprevir 150 mg once daily, and asunaprevir 100 mg twice daily. Patients with a uRVR were treated for 3 weeks. Patients who did not achieve a uRVR were switched to sofosbuvir and ledipasvir for either 8 weeks or 12 weeks and followed up (although not included in subsequent studies). A plasma HCV RNA threshold of less than 500 IU/mL by day 2 was chosen because in China this is the most commonly used initial screening criteria, although more sensitive assays are available. Additionally, our preliminary data (not shown) suggested that using this threshold at 48 h was a surrogate for SVR12 with 3 weeks of pan-oral direct-acting antiviral therapy, irrespective of previous interferon-based therapy response.

The primary endpoint was the proportion of subjects with a sustained virologic response at 12 weeks after treatment completion (SVR12), as defined by HCV RNA below the lower limit of quantification (25 IU/mL). The main safety endpoint was the frequency and severity of adverse events. Secondary endpoints were: the proportion of patients with undetectable HCV viral load at specified timepoints during treatment (day 2, day 4, day 7, week 2, and week 3) and after treatment (week 4 and week 12); the kinetics of circulating HCV RNA from baseline; the proportion of patients with adverse events; discontinuation rates related to adverse events; safety laboratory changes; and the occurrence of HCV resistance mutations. Plasma HCV RNA levels at 0, 1, 2, 4, 8, and 24 hours, and days 2, 4, 7, 14 and 21, were measured for viral kinetic modeling.

Results

RVR was achieved in 18 (66.7%) patients (6/12, 6/6, 6/8 for group 1, 2, 3 respectively, p=0.06). Baseline viral load (V₀) was significantly lower in those with RVR as compared to those without RVR (log₁₀ V₀ 5.81 vs 7.23, p<0.0001). All 18 subjects with RVR were given three weeks of triple therapy and achieved SVR12 with no significant adverse events.

Conclusions

In this proof-of-concept study, all non-cirrhotic Chinese subjects with chronic HCV genotype 1b infection, and who achieved RVR on three potent triple-DAAs regimens containing NS3, NS5A and NS5B inhibitors by Day 2, and receiving a total of three weeks of treatment were cured with excellent tolerability. (ClinicalTrials.gov number NCT02470858).

Methods

Participants

A total of 27 individuals were screened, and 26 of them were enrolled, in an exploratory cohort to assess safety and efficacy. Subjects were enrolled at one center in China (Humanity and Health Medical Centre, Hong Kong SAR). Key inclusion criteria were: male and female, >18 years of age; documented chronic HCV genotype 1b infection for more than six months; a baseline plasma HCV RNA concentration between 10⁴ and 10⁷ IU/mL; absence of cirrhosis as assessed with liver biopsy or by FibroScan®<12.5 kPa. Written informed consent was obtained from all subjects.

Study Oversight

This study was approved by the independent ethics committee at the study center and was conducted in compliance with the Declaration of Helsinki, Good Clinical Practice guidelines, and local regulatory requirements. The study was designed and conducted by the collaborators/investigators (G. Lau, Y. Benhamou and R. F. Schinazi). The investigators collected the data, monitored the study and performed the statistical analyses. An independent data and a safety monitoring committee reviewed the progress of the study. The investigators and participating institution agreed to maintain confidentiality of the data. All the authors had access to the data and assume responsibility for the integrity and completeness of the reported data. The full protocol is included in the appendix.

Study Design

This was an open labeled, proof-of-concept, Phase 2a study conducted at a single-center. Twenty-six eligible subjects were randomized to receive SOF, LDV and ASV (group 1; N=12), SOF, DCV and SMV (group 2; N=6), or SOF, DCV and ASV (group 3; N=8). The administered doses were as follows: SOF 400 mg QD; LDV 90 mg QD; DCV 60 mg QD; SMV 150 mg QD and ASV 100 mg BID. Eighteen subjects with 6 in each of the three treatment groups, who achieved an on treatment plasma HCV RNA <500 IU/mL by day 2, were treated for a total of 3 weeks. Subjects who failed to achieve plasma HCV RNA <500 IU/mL by day 2 were switched at day 3 to SOF and LDV for a total duration of 8 or 12 weeks (FIG. 1). Plasma HCV RNA <500 IU/ml was chosen as in the real world this is the lower limit of detection being used. In addition, our preliminary data (not shown) suggested that using this level as the threshold by 48 hours translated to SVR12 with three weeks pan-oral DAA therapy.

Efficacy and Safety Assessments

Screening assessments included measurement of the plasma HCV RNA level, IL28B genotype with the use of a TaqMan genotyping assay (Applied Biosystems) for the rs12979860 single-nucleotide polymorphism, and IFNL4 (rs368234815) genotyping and standard laboratory tests. Plasma HCV RNA concentration was measured at baseline (0 hr), 1, 2, 4, 8, 24 hr after initial dosing and at days 2, 4, 7, 14 and 21 or end of treatment. After the end-of-treatment, all subjects had plasma HCV RNA measured at 4 week intervals until week 12. Plasma HCV RNA concentrations were measured by COBAS TaqMan 48 analyzer, version 2.0 (Roche Molecular Systems), with a lower limit of quantification of 25 IU/mL and a lower limit of detection of 6 IU/mL. HCV genotype and subtype were determined at screening using the Versant HCV Genotype INNOLiPA 2.0 assay (Siemens Healthcare Diagnostics). Deep sequencing of the NS5A and NS5B regions of the HCV RNA was performed in all subjects at baseline, and again at the time of failure in those who had virologic failure (Kai et al., Emergence of hepatitis C virus NS5A L31V plus Y93H variant upon treatment failure of daclatasvir and asunaprevir is relatively resistant to ledipasvir and NS5B polymerase nucleotide inhibitor GS-558093 in human hepatocyte chimeric mice. J Gastroenterol 2015; 50: 1145-51).

Virological failure resulting in study drug discontinuation was defined as: failure to achieve plasma HCV RNA concentrations less than 25 IU/mL during the 3 weeks of therapy; confirmed HCV RNA concentrations of 25 IU/mL or more at two consecutive measurements at any point in patients with on-treatment HCV RNA concentrations less than 25 IU/mL; or a confirmed increase in HCV RNA at two consecutive measurements of greater than 1 log₁₀ IU/mL above the nadir at any timepoint during treatment. Liver stiffness was measured with FibroScan (Echosens, Paris, France) according to the manufacturer's instructions.

The resulting sequences were compared to detect resistance-associated variants that emerged during treatment. Resistance-associated variants that were present in more than 1% of sequence reads are reported herein.

Vital signs and symptom-directed physical examinations and adverse events graded according to the NIAID Division of AIDS toxicity table (version 1.0) were recorded at baseline, D2, D4, D7, week 2, and week 3 during treatment, and then at weeks 4 and 12 after end of treatment. Blood was taken for hematology and chemistry at screening, baseline, D2, D4, D7, week 2 and week 3 during treatment, and then at weeks 4 and 12 after end of treatment. 12-lead electrocardiogram was performed at screening, baseline, week 2 and week 3 during treatment, and then at weeks 4 and 12 after end of treatment.

Plasma samples from patients infected with HCV were stored in aliquots at −80° C. until further processing. Viral RNA was extracted and the genome sequenced using an Applied Biosystems 3730xl DNA analyser as previously described.²¹ Data were analysed with the ABL-DeepChek-HCV 1.4 software (ABL SA, Luxembourg, Luxembourg). To confirm compliance, a liquid chromatography-tandem mass spectrometry (LC-MS/MS) method was developed to detect and quantify the anti-HCV drug concentrations in plasma collected from each patient. Briefly, blood samples were collected from 25 patients at 8 h, 24 h, and 48 h after initiating treatment. 50 μL of plasma was extracted with 3 mL of ethyl acetate containing abacavir (as an internal standard). The supernatant was collected, air dried, then reconstituted in 2 mmol/L ammonium acetate solution with 75% methanol. Five target compounds were simultaneously monitored and quantified by LC-MS/MS in the multiple reaction monitoring mode, including sofosbuvir (m/z 530.2/243.2), daclatasvir (m/z 739.5/339.3), ledipasvir (m/z 889.4/637.3), asunaprevir (m/z 748.4/648.4), and simeprevir (m/z 750.4/315.2). A Dionex Ultimate 3000 HPLC system (Thermo Scientific, Waltham, Mass., USA) coupled with an AB SCIEX API5000 triple quadrupole mass spectrometer (AB SCIEX, Framingham, Mass., USA) was used for analysis.

Virologic Failure, Viral Breakthrough, and Resistance Monitoring

Per protocol, virologic failure resulting in study drug discontinuation was defined as: (1) failure to achieve plasma HCV RNA <25 IU/mL by 2 days or (2) confirmed HCV RNA ≥25 IU/mL at 2 consecutive measurements at any point in subjects with on treatment HCV RNA <25 IU/mL or (3) confirmed increase in HCV RNA at 2 consecutive measurements of >1 log₁₀ IU/mL above nadir at any time point during treatment.

End Points

The primary efficacy endpoint was the proportion of individuals with a SVR at 12 weeks after the end of treatment as defined by HCV RNA below the lower limit of quantification (25 IU/mL). The main safety endpoint was the frequency and severity of adverse events. Secondary endpoints were: (1) the proportion of subjects with undetectable HCV viral load at specified time points during and after treatment; (2) the proportion of patients with adverse events; (3) discontinuation rates related to adverse events; (4) safety laboratory changes, (5) the kinetics of circulating HCV RNA from baseline; and (6) the occurrence of HCV-resistance mutations.

Statistical Analysis

A minimum of 14 patients were needed to power this proof-of-concept clinical study and therefore guide the decision for comparative late-stage clinical studies. A sample size of 18 patients, with six in each treatment group, was selected to meet power calculation requirements. Assuming at least a 1% increase in SVR12 rate in each group from the literature, with a sample size of six in each group, the power to detect such an increase ranged from 78% to 99% at the a level of 0.05, depending on the magnitude of the increase.

Baseline characteristics between groups were compared using the Kruskal-Wallis test for continuous outcomes and Fisher's exact tests for binary outcomes. The difference in HCV viral load decline between groups was compared using Kruskal-Wallis test and corrected by Dunn's test for multiple comparison between groups.

HCV RNA concentrations and liver stiffness before treatment, at the end of treatment, and 12 weeks after treatment were compared using Wilcoxon signed-rank test for each group. Statistical analysis was based on the intention-to-treat principle. Since this was a phase 2a proof-of-concept study, the small sample size did not allow the analysis of predictive factors for uRVR using a multivariate model.

Significance level was set to p<0.05. Statistical analyses were done with Stata (release 13; StataCorp). Viral kinetic modelling was done with a multiscale model (Guedj J, Dahari H, Rong L, et al. Modeling shows that the NS5A inhibitor daclatasvir has two modes of action and yields a shorter estimate of the hepatitis C virus half-life. Proc Natl Acad Sci USA 2013; 110: 3991-96; Rong et al. Analysis of hepatitis C virus decline during treatment with the protease inhibitor danoprevir using a multiscale model. PLoS Comput Biol 2013; 9: e1002959) for all 26 participants (appendix p 12).

Results

Subjects

Between Feb. 15, 2015, and Mar. 12, 2015, 27 subjects with chronic hepatitis C, were assessed for eligibility. One subject with cirrhosis was excluded and 26 were randomized in the three treatment groups (FIG. 1). Six of 12 (50%, 95% CI 25-75) patients receiving sofosbuvir, ledipasvir, and asunaprevir, six of six (100%, 61-100) receiving sofosbuvir, daclatasvir, and simeprevir, and six of eight (75%, 41-95) receiving sofosbuvir, daclatasvir, and asunaprevir achieved plasma HCV RNA concentrations less than 500 IU/mL by day 2, and subsequently received triple direct-acting antiviral therapy for 3 weeks (ρ=0.10). The baseline characteristics of the enrolled persons are shown in Table 1.

TABLE 1
Baseline demographic and clinical characteristics of the
patients who achieved an ultrarapid virological response

| | Sofosbuvir, | | Sofosbuvir, | | Sofosbuvir, | |
|---|---|---|---|---|---|---|
| | ledipasvir, | | daclatasvir, | | daclatasvir, | |
| | and | | and | | and | |
| | asunaprevir | | simprevir | | asunaprevir | |
| | 3 weeks | | 3 weeks, | | 3 weeks | |
| | (n = 6) | | (n = 6) | | (n = 6) | |
| Age (years [mean, range]) | 41 | (25-66) | 40 | (23-59) | 31 | (21-47) |
| Sex | | | | | | |
| Male | 2 | (33%) | 2 | (33%) | 2 | (33%) |
| Female | 4 | (67%) | 4 | (67%) | 4 | (67%) |
| BMI (kg/m² [mean, range]) | 23.7 | (4.5) | 21.1 | (2.6) | 19.2 | (2.0) |
| HCV RNA (log10 IU/mL) | | | | | | |
| Mean (SD) | 6.3 | (0.3) | 5.7 | (1.0) | 5.9 | (0.8) |
| Median (IQR) | 6.2 | (6.1-6.6) | 5.6 | (5.3-6.7) | 5.9 | (5.2-6.5) |
| HCV RNA ≥ 800 000 IU/mL | 5 | (83%) | 2 | (33%) | 3 | (50%) |
| Previous treatment | | | | | | |
| Treatment | 3 | (50%) | 2 | (33%) | 1 | (17%) |
| experienced | | | | | | |
| Treatment naive | 3 | (50%) | 4 | (67%) | 5 | (83%) |
| Previous response* | | | | | | |
| Relapser | 1 | (33%) | 2 | (100%) | 1 | (100%) |
| Partial responder | 1 | (33%) | 0 | | 0 | |
| Null responder | 1 | (33%) | 0 | | 0 | |
| IL28B genotype | | | | | | |
| CC | 4 | (67%) | 4 | (67%) | 3 | (50%) |
| CT | 2 | (33%) | 2 | (33%) | 3 | (50%) |
| IFNL4 genotype | | | | | | |
| TT/TT | 4 | (67%) | 4 | (67%) | 5 | (83%) |
| ΔG/TT | 2 | (33%) | 2 | (33%) | 1 | (17%) |
| Liver stiffness | 6.1 | (1.7) | 5.8 | (2.0) | 5.5 | (0.9) |
| measure (kPa) | | | | | | |
| Fibrosis stage | | | | | | |
| (METAVIR)† | | | | | | |
| F0-F1 (LSM ≤ 7.0 kPa) | 4 | (67%) | 4 | (67%) | 6 | (100%) |
| F2 (7.0 kPa < LSM ≤ | 2 | (33%) | 2 | (33%) | | |
| 9.5 kPa) | | | | | | |
| F3 (9.5 kPa < LSM ≤ | 0 | | 0 | | 0 | |
| 12.5 kPa) | | | | | | |

Data are mean (SD) or n (%), unless otherwise stated. BMI = body-mass
index. HCV = hepatitis C virus. *Data indicate the response to the most
recent previous regimen: a relapser is a patient who received at least 36
weeks of pegylated interferon and ribavirin with HCV DNA undetectable at
the end of treatment, but detectable within 52 weeks of follow-up; a partial
responder is a patient who received at least 20 weeks of pegylated interferon
and ribavirin for the treatment of HCV and achieved ≥2 log10 IU/mL
reduction in HCV RNA at week 12, but failed to achieve HCV RNA
undetectable at the end of treatment; a null responder is a patient who
received at least 12 weeks of pegylated interferon and ribavirin and failed to
achieve a 2 log10 IU/mL reduction in HCV RNA at week 12. †Liver stiffness
was determined by Fibroscan; no patient in this study had cirrhosis (fibrosis
score >12.5 kPa).

| | |
|---|---|
| Baseline | |
| Characteristics of | |
| patients who did | |
| not achieve uRVR | |
| HCV RNA >= 500 | |
| TU/mL by day 2 | |
| SOF + LDV | |
| 8-12 weeks | P |
| n = 8 | value |

| | |
|---|---|
| Age -- yr | 0.84 |

| | |
|---|---|
| Mean | 36 |
| Range | 21-50 |

| | | | |
|---|---|---|---|
| Male sex -- no. (%) | 4 | (50) | 0.35 |
| ^{#}BMI (kg/m²) | | | 0.06 |

| | | |
|---|---|---|
| Mean | 23.7 | |
| Range | 18.5-28.3 | |
| HCV RNA -- Log10 | 7.0 ± 0.3 | <0.001 |
| IU/mL | | |

| | | | |
|---|---|---|---|
| HCV RNA ≥ 800000 | 8 | (100) | 0.03 |
| IU/mL -- no. (%) | | | |

| | |
|---|---|
| Previous treatment -- no. (%) | 0.22 |

| | | |
|---|---|---|
| Experienced | 5 | (62.5) |
| Naïve | 3 | (37.5) |

| | |
|---|---|
| ^{&}Previous response -- no. (%) | 0.99 |

| | | |
|---|---|---|
| Relapser | 3 | (60.0) |
| Partial response | 2 | (40.0) |
| Null response | 0 | (0) |

| | |
|---|---|
| IL28B genotype -- no. (%) | 0.67 |

| | | |
|---|---|---|
| CC | 6 | (75.0) |
| CT | 2 | (25.0) |

| | |
|---|---|
| IFNL4 genotype -- no. (%) | 0.99 |

| | | |
|---|---|---|
| TT/TT | 6 | (75.0) |
| ΔG/TT | 2 | (25.0) |

| | | |
|---|---|---|
| Fibrosis score | 7.3 ± 2.5 | 0.10 |
| ^{$} Fibrosis stage | 0.11 | |
| (METAVIR) -- no. (%) | | |

| | | |
|---|---|---|
| F0-F1 (Fibroscan | 5 | (62.5) |
| score <= 7.3) | | |
| F2 (7.3 < Fibroscan | 1 | (12.5) |
| score <= 9.7) | | |
| F3 (9.7 < Fibroscan | 2 | (25.0) |
| score <= 14.7) | | |

Data are mean (SD) or n (%) unless otherwise stated.
*Plus-minus values are mean ± SD. The Kruskal-Wallis test and Fisher's exact test were used to test for the difference across treatment groups for continuous and categorical variables respectively. There were no significant differences among the treatment groups for all variables except for BMI (p = 0.047). HCV denotes hepatitis C virus, SOF sofosbuvir, DCV daclatasvir, LDV ledipasvir, SMV simeprevir, ASV asunaprevir.
^{#}The body-mass index (BMI) is the weight in kilograms divided by the square of the height in meters.
^{&}Data indicate the response to the most recent PR-based regimen.

Relapser is the subject who received at least 36 weeks of pegIFN/RBV for the treatment of HCV and was undetectable at the end of treatment, but HCV RNA was detectable within 52 weeks of treatment follow-up.

Partial responder is the subject who received at least 20 weeks of pegIFN/RBV for the treatment of HCV and achieved ≥2 log 10 IU/mL reduction in HCV RNA at Week 12, but failed to achieve HCV RNA undetectable at the end of treatment.

Null responder is the subject who received at least 12 weeks of pegIFN/RBV for the treatment of HCV and failed to achieve a 2 log 10 IU/mL reduction in HCV RNA at Week 12.

^{$} The liver stiffness is determined by Fibroscan (Echosens, France). No patient in this study had cirrhosis (Fibrosis score >12.5 kPa).

All participants were Chinese, six (33%) were male, seven (39%) had the IL28 non-CC genotype, and eight (44%) had baseline HCV RNA less than 800 000 IU/mL. Four (22%) patients had stage 2 liver fibrosis. Patients in each of the 3-week treatment groups were well matched except for body-mass index. The eight patients who did not achieve uRVR were switched to sofosbuvir and ledipasvir by day 3 and followed up, but were not included in subsequent studies, and hence had a choice of duration of therapy of 8 weeks (n=4) or 12 weeks (n=4). Patients with uRVR had a significantly lower mean baseline HCV RNA than those without uRVR (7.0 log₁₀ IU/mL [95% CI 6.8-7.2] vs 6.0 log₁₀ IU/mL [5.6-6.3], ρ<0.0001; appendix p 2). The eight patients without uRVR were not considered in the efficacy and safety analyses. However, none of them had grade 3-4 adverse events and all of these patients achieved SVR12.

No difference regarding demographic, biological and virologic findings at baseline was observed between subjects with plasma HCV RNA ≥500 IU/mL and <500 IU/mL by day 2 except for baseline HCV RNA level (Table 1).

Efficacy

At week 3, all 18 patients who achieved uRVR had undetectable plasma HCV RNA irrespective of the allocated direct-acting antiviral regimen. The median time to achieve plasma HCV RNA less than 25 IU/mL was shorter in patients receiving sofosbuvir, ledipasvir, and asunaprevir than in patients receiving sofosbuvir, daclatasvir, and asunaprevir (4 days [IQR 0] vs 14 days [7]; ρ=0.01). Plasma HCV RNA remained below the limit of quantification up to week 12 after completion of treatment (table 2). The median time to achieve plasma HCV RNA less than 25 IU/mL was significantly shorter in patients who achieved uRVR (7 days [IQR 10]) than in those who did not achieve this threshold (17.5 days [10.5]; ρ=0.003; Table 2).

TABLE 2
Proportion of subjects with HCV RNA level lower than the quantification limit*

| Lower than the quantification limit | | | | | |
|---|---|---|---|---|---|
| SOF + LDV + ASV | | SOF + DCV + SMV | | SOF + DCV + ASV | |
| (Group 1) | | (Group 2) | | (Group 3) | |
| n = 6 | | n = 6 | | n = 6 | |
| n | % (95% CI) | n | % (95% CI) | n | % (95% CI) |

During 3-wk treatment period

| | | | | | | |
|---|---|---|---|---|---|---|
| Day 2 | 1 | 16.7 (0.9-81.4) | 2 | 33.3 (4.2-85.1) | 0 | 0 |
| Day 4 | 5 | 83.3 (18.6-99.1) | 2 | 33.3 (4.2-85.1) | 0 | 0 |
| Day 7 | 6 | 100 | 4 | 66.7 (14.9-95.8) | 2 | 33.3 (4.2-85.1) |
| Week 2 | 6 | 100 | 6 | 100 | 5 | 83.3 (18.6-99.1) |
| Week 3 | 6 | 100 | 6 | 100 | 6 | 100 |

During post-treatment period

| | | | | | | |
|---|---|---|---|---|---|---|
| At week 4 | 6 | 100 | 6 | 100 | 6 | 100 |
| At week 12 | 6 | 100 | 6 | 100 | 6 | 100 |

*The limit of quantification for plasma HCV RNA by Roche COBAS Taqman test was 25 IU/mL.
p = 0.01 for the difference in the median time for HCV RNA level to be lower than the quantification limit between SOF + LDV + ASV and SOF + DCV + ASV. Significance was tested by Kruskal-Wallis test and corrected by Dunn's test for multiple comparison between groups.

A post-hoc analysis of liver stiffness showed a significant decrease from baseline to 12 weeks post-treatment in patients receiving sofosbuvir, ledipasvir, and asunaprevir (6.1 kPa vs 4.9 kPa, ρ=0.028) and in patients receiving sofosbuvir, daclatasvir, and simeprevir (5.8 kPa vs 5.1 kPa, ρ=0.046), but not in patients receiving sofosbuvir, daclatasvir, and asunaprevir (5.5 kPa vs 5.4 kPa, ρ=0.53).

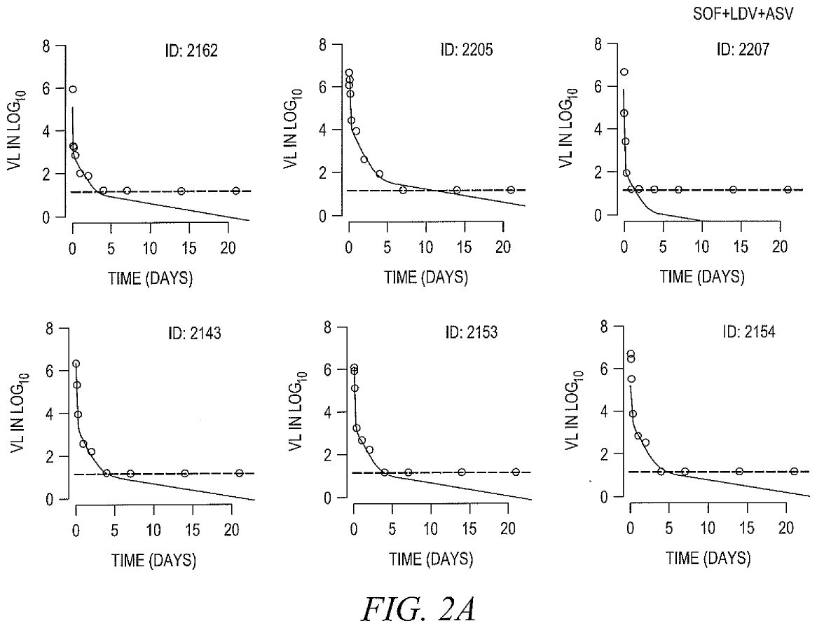

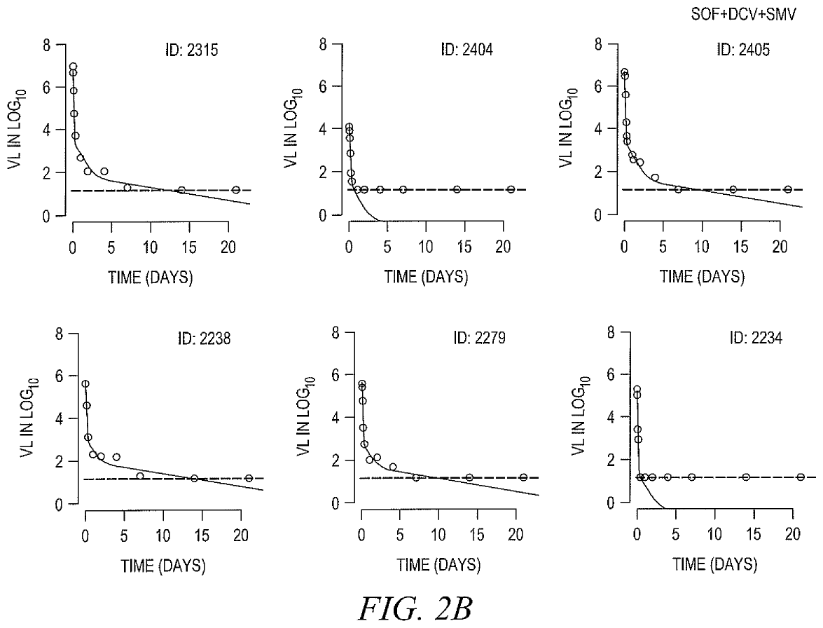

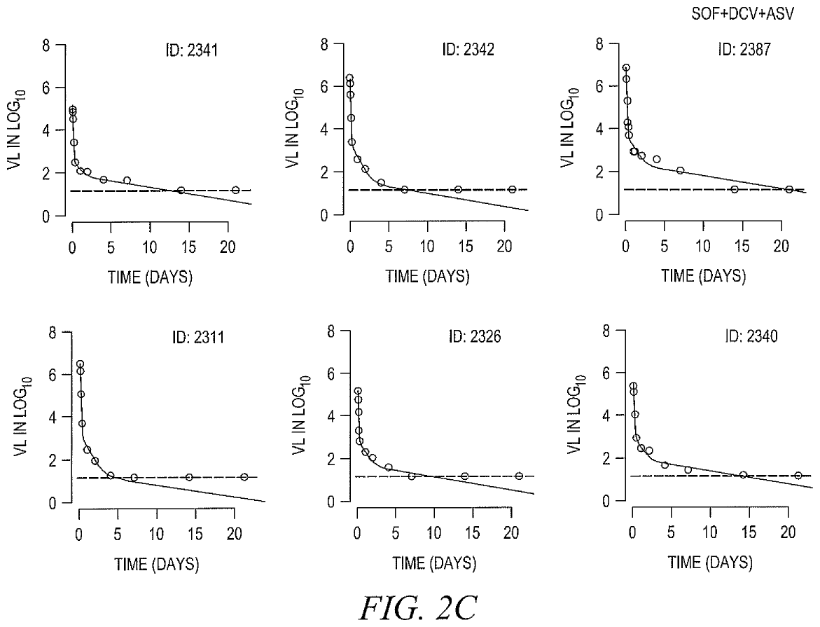

The effect of overall treatment on HCV clearance was estimated by taking into account the effectiveness of the regimen in inhibiting HCV RNA synthesis (ε_{α}) and virion secretion or export (εₛ), with ε=1 being 100% effective and enhancing intracellular HCV RNA decay (by a factor κ). The model fit individual patient data well as shown in the appendix (p 9). HCV RNA kinetics exhibit a three-phase viral decline, with a rapid first phase, moderate decay during an intermediate phase, and then a slower third phase. No significant difference was recorded in the decline rate of the mean (FIG. 2) and median HCV load between the three treatment groups (appendix p 8). Based on 18 patients with uRVR, the estimated mean effectiveness of the treatments was εα=0.9962 and εₛ=0.9987 (appendix p 7). The mean estimated rate of viral clearance was 29.7 per day and the mean effectiveness of the direct-acting antiviral therapy in enhancing intracellular HCV RNA degradation, κ, was 1.49, consistent with previous estimates.²² Using data from all 26 treated patients, parameter values were not distributed differently among patients with uRVR and those without uRVR (data not shown). However, the estimated baseline viral load (V0) was significantly lower in those with uRVR than in those without uRVR (log₁₀ V0 5.81 vs 7.23, ρ<0.0001; appendix p 11).

Resistance-associated variant analysis was done at baseline to elucidate the naturally occurring resistance profile. Since virus clearance was achieved rapidly in all patients during the treatment, it was difficult to do resistance-associated variant analysis at other timepoints. Sequencing of NS3/4A, NS5A, and NS5B was successful in all 26 baseline samples. Five naturally occurring protease inhibitor resistance-associated variants (V36L, T54S, S122R, I132V, or D168H/N/Y) were identified in NS3/4A in 23 (88%) of 26 baseline samples. The I132V variant was found in all 23 samples and among them, five had one additional mutation (two D168H/N/Y, one S122R, one T54S, and one V36L). Four NS5A variants associated with resistance to daclatasvir or ledipasvir (Q30R, L31F/V, H54N, or Y93H) were identified in 23 (88%) of 26 samples.

The Q30R variant was found in all 23 samples and five had one or two additional mutations (appendix p 13). Other minor mutations are summarized in the appendix (p 13). None had the 282T mutation associated with sofosbuvir resistance.

Plasma drug concentrations provided a confirmation of drug delivery and patient compliance during the study (appendix pp 14, 15). Drugs taken by patients were consistent with their regimen, with only a few exceptions noted in which plasma drug concentrations were lower than the limit that could be quantified by HPLC-MS.

The median time to achieve plasma HCV RNA <25 IU/mL was significantly shorter in subjects who achieved Day 2 HCV RNA <500 IU/mL than in those who did not achieve this threshold (ρ=0.003, Table 3).

TABLE 3
Proportion of individuals with HCV RNA level
lower than the quantification limit*

| Fast responders | | Slow responders | |
|---|---|---|---|
| (plasma HCV | | (plasma HCV | |
| RNA < 500 IU/mL | | RNA ≥ 500 IU/mL | |
| by Day 2) | | by Day 2) | |
| N = 18 | | N = 8 | |
| n | % (95% CI) | n | % (95% CI) |

During treatment period

| | | | | |
|---|---|---|---|---|
| Day 2 | 3 | 16.7 (5.0-43.3) | 0 | 0 |
| Day 4 | 7 | 38.9 (18.6-63.9) | 0 | 0 |
| Day 7 | 12 | 66.7 (40.9-85.2) | 1 | 12.5 (1.3-60.5) |
| Week 2 | 17 | 94.4 (65.7-99.3) | 4 | 50 (17.4-82.6) |
| Week 3 | 18 | 100 | 6 | 75 (33.2-94.8) |
| Week 4 | na | na | 8 | 100 |
| Week 8 | na | na | 8 | 100 |
| Week 12 | na | na | 8 | 100 |

During post-treatment period

| | | | | |
|---|---|---|---|---|
| At week 4 | 18 | 100 | 8 | 100 |
| At week 12 | 18 | 100 | 8 | 100 |

*The limit of quantification for plasma HCV RNA by Roche COBAS Taqman test was 25 IU/mL.
p = 0.003 for the difference in the median time for HCV RNA level to be lower than the quantification limit between these two groups. Significance was tested by Wilcoxon-Mann-Whitney test.
na = not applicable

Viral Kinetic Study

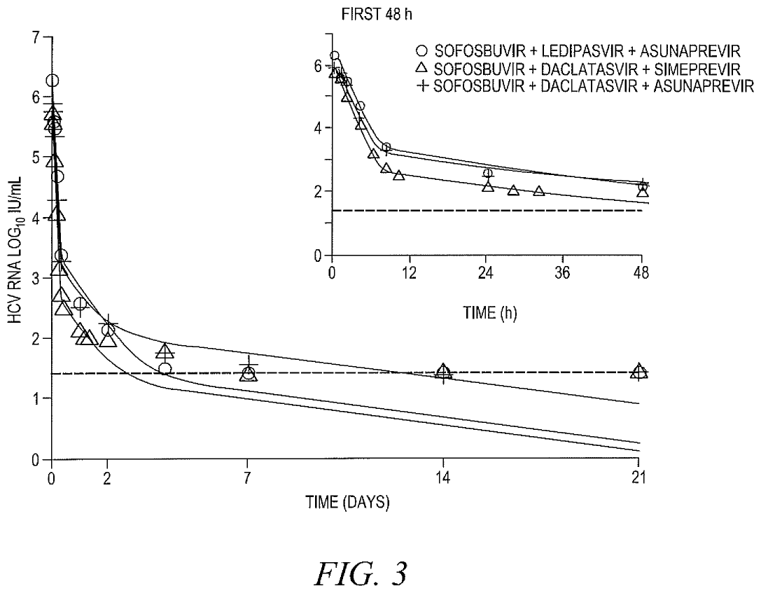

Prior in vitro studies and modeling of in vivo data have shown that both NS5A and HCV protease inhibitors can inhibit both HCV RNA replication and virion assembly, maturation or secretion^{26,27,28,29}. Using our model, the effect of overall treatment on HCV clearance was estimated by taking into account the effectiveness of the regimen in inhibiting HCV RNA synthesis and virion secretion/export (ε_{α} and εₛ, respectively) and enhancing intracellular HCV RNA decay (x). The model fit individual patient data well as shown in FIGS. 2 and 3.

In FIG. 3, Symbols show the mean log 10 viral load for each treatment group, whereas the solid lines show the mean model predicted trajectories, which were calculated from the predicted viral load, Eq. (1) with the estimated parameters given in Table 7 for each patient in a group and then taking the mean. The dashed horizontal line indicates the assay lower limit of quantification. There appears to be three phases in the viral decline in all three treatment groups. The trend was more apparent in treatment group SOF+DCV+SMV (group 2). Note, in calculating the mean of the data, the lower limit of quantification was used for data below the limit, so that the data symbols on the horizontal line denote points where the data for all 6 individuals in the treatment group had unquantifiable HCV RNA.

FIG. 3 plots the decline of the median viral load and avoids the bias introduced by data below the quantification limit. The baseline viral loads were different in the three groups giving the appearance of different viral decline kinetics (see inset), although no significant difference was observed in the viral decline rate between these three treatment groups. HCV denotes hepatitis C virus, SOF sofosbuvir, DCV daclatasvir, LDV ledipasvir, SMV simeprevir, ASV asunaprevir.

The HCV RNA kinetics exhibit a three-phase viral decline, with a rapid first phase, moderate decay during an intermediate phase, and then a slower third phase. No significant difference was observed in the decline rate of the mean (FIG. 2) and median hepatitis C viral load between these three treatment groups (FIG. 3). Based on 18 subjects with RVR, the estimated mean efficacy of the treatments are ε_{α}=0.9962, εₛ=0.9987, respectively (Tables 5 and 6). The mean estimated rate of viral clearance is 29.7 day⁻¹ and the mean effectiveness of the DAAs in enhancing intracellular HCV RNA degradation, x, is 1.49, consistent with previous estimates.26 Using data from all 26 treated subjects, parameter values were not found to be distributed differently among patients with RVR and those without RVR. However, the baseline viral load (V₀) was significantly lower in those with RVR as compared to those without RVR (log₁₀ V₀ 5.81 vs. 7.23, ρ<0.0001, Tables 8 and 9).

Safety

Of the 18 patients who completed treatment, the most common adverse events were headache and fatigue (table 3), and all adverse events were mild to moderate. Two patients had grade 3 laboratory abnormalities: one had anaemia due to menorrhagia related to uterine fibroids and another had transient hyperbilirubinaemia. The cause of the transient hyperbilirubinaemia is unknown. Previously, this adverse event was also reported in a clinical study of daclatasvir plus asunaprevir. In the instant study, it was only recorded in one of six patients receiving sofosbuvir, ledipasvir, and asunaprevir. No grade 4 laboratory abnormalities were reported.

TABLE 4
Adverse Events and Laboratory Abnormalities during the Treatment Period

| | SOF + | SOF + | SOF + |
|---|---|---|---|
| | LDV + ASV | DCV + SMV | DCV + ASV |
| | (Group 1) | (Group 2) | (Group 3) |
| | n = 6 | n = 6 | n = 6 |
| Adverse event | | | |
| Any serious adverse event during treatment | 0 | 0 | 0 |
| Any adverse event leading to | 0 | 0 | 0 |
| discontinuation of study drug - no. of | | | |
| subjects (%) | | | |
| Common adverse events | | | |
| Fatigue | 1 (17%) | 1 (17%) | 2 (33%) |
| Nausea | 1 (17%) | 0 | 0 |
| Headache | 1 (17%) | 1 (17%) | 1 (17%) |
| Dizziness | 0 | 1 (17%) | 0 |
| Insomnia | 1 (17%) | 0 | 1 (17%) |
| Abdominal pain | 0 | 1 (17%) | 1 (17%) |
| Constipation | 1 (17%) | 0 | 0 |
| Diarrhea | 0 | 1 (17%) | 1 (17%) |
| Dermatitis | 0 | 1 (17%) | 1 (17%) |
| Common cold | 1 (17%) | 0 | 1 (17%) |
| Laboratory abnormalities | | | |
| Any grade 3 abnormality | 1 (17%) | 1 (17%) | 1 (17%) |
| during treatment | | | |
| Grade 3 abnormalities | | | |
| Decreased haemoglobin | 0 | 1 (17%) | 0 |
| Raised total bilirubin | 1 (17%) | 0 | 0 |

There were no deaths or discontinuations due to adverse events. All adverse events and serious adverse events were recorded from the time the consent form was signed through 14 days after cessation of treatment. The relatedness (probable or possible) of the adverse event to the regimen was determined by the investigator; subjects could have had more than one adverse event.

There were no disruptions or discontinuations of study treatment due to any adverse events. Two subjects had grade 3 laboratory abnormalities-one anemia due to menorrhagia related to uterine fibroid and another transient hyperbilirubinemia with cause unknown. No grade 4 laboratory abnormalities were reported.

Discussion

Discussion

In this exploratory cohort of Chinese patients with chronic genotype 1b HCV who had no cirrhosis and had a uRVR to triple direct-acting antiviral regimens, we show that these regimens are well tolerated and a 100% cure rate is achievable. This study reports the shortest treatment duration of pan-oral direct-acting antivirals used for patients with chronic HCV infection. The incidence of adverse events was low compared with other reported studies on pan-oral direct-acting antiviral agents, which might be related to the shorter duration of therapy. The patients have been followed up, and no relapse or adverse events have been recorded as approximately ten months from the end of the trial.

Current recommendations for genotype 1b patients without cirrhosis include: 8-12-week therapy with ledipasvir and sofosbuvir; or 12-week therapy with paritaprevir with ritonavir plus ombitasvir and dasabuvir, or sofosbuvir plus simeprevir, or sofosbuvir plus daclatasvir, or grazoprevir (MK-5172) and elbasvir (MK-8742), yielding an SVR12 in more than 95% of recipients (Omata, et al. APASL consensus statements and recommendation on treatment of hepatitis C. Hepatol Intl 2016; published online April 29. DOI:10.1007/s12072-016-9717-6).

A major factor restricting the wide availability of direct-acting antivirals to patients with HCV is the high cost of treatment, about US$200-1200 per day, even when discounted. Several clinical trials were designed to shorten treatment duration to 6-8 weeks, with more than an 80-90% success rate (Kohli, et al. Virological response after 6 week triple-drug regimens for hepatitis C: a proof-of-concept phase 2A cohort study. Lancet 2015; 385: 1107-13; Sulkowski et al. Efficacy and safety of 8 weeks versus 12 weeks of treatment with grazoprevir (MK-5172) and elbasvir (MK-8742) with or without ribavirin in patients with hepatitis C virus genotype 1 mono-infection and HIV/hepatitis C virus co infection (C-WORTHY): a randomised, open-label phase 2 trial. Lancet 2015; 385: 1087-97).

Attempts to further shorten therapy to 4 weeks have so far failed. In the C-SWIFT genotype 1 study (Poordad, et al. C-swift: grazoprevir/elbasvir+sofosbuvir in cirrhotic and noncirrhotic, treatment-naive patients with hepatitis C virus genotype 1 infection, for durations of 4, 6 or 8 weeks and genotype 3 infection for durations of 8 or 12 weeks. J Hepatol 2015; 62: S192-93), only three of the five patients with genotype 1b chronic hepatitis C without cirrhosis achieved an SVR at 4 weeks or 8 weeks after the end of therapy with a 4-week fixed-dose combination of grazoprevir (MK-5172), elbasvir (MK-8742), and sofosbuvir 400 mg once daily. Similarly, in the NIH/UMD Synergy trial (Kattakuzhy, et al. J Hepatol 2015; 62: S669) of patients with early F0-F2 fibrosis treated with ledipasvir and sofosbuvir plus GS-9451 and GS-9669, only five (20%) of 25 achieved SVR12, whereas ten (40%) of 25 in another cohort treated with ledipasvir and sofosbuvir plus GS-9451 achieved an SVR12.

Modelling of HCV RNA kinetics during anti-HCV drug therapy has predicted faster first phases in protocols using an NS5A inhibitor, such as daclatasvir (Guedj, et al. Proc Natl Acad Sci USA 2013; 110: 3991-96) or ledipasvir (Lawitz, et al. J Hepatol 2012; 57:24-31), and faster second phases using a protease inhibitor (Guedj and Perelson, Hepatology 2011; 53: 1801-08). The present inventors considered that this finding, together with the use of sofosbuvir, could allow a shorter treatment duration. The instant study strongly suggests that combining the three different approved oral anti-HCV drug classes leads to a complete cure (SVR12) in 3 weeks in all patients with genotype 1b HCV without cirrhosis who had plasma HCV RNA reduced to less than 500 IU/mL within the first 2 days of therapy. No differences were observed between the three combinations of the drugs used in both day 2 response and SVR12.

Nevertheless, patients receiving sofosbuvir, ledipasvir, and asunaprevir had shorter time to antiviral efficacy than did patients receiving sofosbuvir, daclatasvir, and asunaprevir; the reason is unclear. The most apparent distinction between these two groups is the inclusion of ledipasvir (sofosbuvir, ledipasvir, and asunaprevir) versus daclatasvir (sofosbuvir, daclatasvir, and asunaprevir). Review of the scientific literature only found one comparison, daclatasvir plus asunaprevir versus sofosbuvir and ledipasvir for hepatitis C genotype 1 in Japanese patients, but this was an indirect comparison (Swallow, et al., J Comp Eff Res 2016; 5: 273-79). No direct comparison of ledipasvir with daclatasvir has been done.

A substantial proportion of the enrolled individuals (18 [69%] of 26) achieved a very rapid and profound drop in plasma HCV RNA. This rapid viral decline can be affected by the presence of baseline NS5A resistance-associated variants (appendix p 13). The reasons for the naturally-occurring resistance-associated variants are still unknown. However, in our study, baseline resistance-associated variants did not affect SVR12 in patients, irrespective of whether they had a uRVR. Even for patients who did not have a uRVR, baseline resistance-associated variants did not affect SVR12 in patients treated for longer than 3 weeks (i.e., 8 weeks or 12 weeks).

To our knowledge, this study is the first to use the principle of response-guided therapy with an all direct-acting antiviral regimen to shorten treatment duration, with a high proportion of patients achieving an SVR12. This treatment can greatly reduce duration of therapy and subsequently compliance and cost, and improve the accessibility and affordability of direct-acting antiviral drugs, especially in middle-income and low-income countries. Additionally, this treatment regimen can reduce the emergence of resistance, development of side-effects, and help to curtail the use of counterfeit direct-acting antiviral drugs in some countries, which could be potentially harmful.

Our findings are not consistent with the concept that cure corresponds to an end of treatment viral load below a cure boundary of less than one viral particle in the extracellular body fluid (ie, 15 L), which corresponds to a concentration of about 10⁻⁴ IU/mL.^{13,14,31} (Guedj, et al., Proc Natl Acad Sci USA 2013; 110: 3991-96; Kohli et al. Lancet 2015; 385: 1107-13; Perelson and, Guedj, Nat Rev Gastroenterol Hepatol 2015; 12: 437-45).

The multiscale model predicted that none of the 18 treated patients would reach the cure boundary (appendix p 9). This finding prompts a reconsideration of whether reducing the amount of virus to less than one virion is really necessary to achieve a cure.

This is in keeping with the findings of the SYNERGY and PILOT trials, where some subjects with detectable plasma HCV RNA levels at the end of treatment, had SVR12 (Kohli, et al. Lancet 2015; 385:1107-13.; Svarovskaia et al., Clin Infect Dis 2014; 59:1666-74).

Currently, no proven explanation has been presented for the lack of viral rebound in persons with detectable HCV RNA at end of treatment in these studies. However, two possibilities exist: first, the immune system controls the virus, which results in a functional cure, as reported in rare instances in persons infected with HIV who ceased therapy (Kohli, et al. Lancet 2015; 385:1107-13.; Yukl, et al. PLoS Pathog 2013; 9:e1003347; Saez-Cirion, et al., ANRS VISCONTI Study. PLoS Pathog 2013; 9:e1003211; Conway and Perelson, Proc Natl Acad Sci USA 2015; 112:5467-72) or second, that the HCV RNA detected by the high sensitivity assay was not infectious.

Given the potential importance of the immune system in the control of viral infection, immunological data, such as anti-HCV antibody levels and levels of cytokines, such as C-X-C motif chemokine 10 (also known as interferon γ-induced protein 10) levels, can be measured to see if they could help improve SVR prediction. Lin, et al. J Infect Dis 2014; 210:1881-5; Rehermann, Nat Med 2013; 19:859-68).

Direct-acting antiviral-mediated clearance of HCV is associated with loss of intrahepatic immune activation by IFN-α, which is indicated by decreased levels of CXCL10 and CXCL11 and normalization of NK cell phenotype and function (Rehermann, Nat Med 2013; 19:859-68). Interestingly, HCV NS3/4A protease inhibitors might also restore innate immune responses within infected cells and these responses can contribute to a rapid loss of intracellular viral RNA. Indeed, it has been shown that HCV NS3/4A protease can efficiently cleave and inactivate two important signaling molecules in the sensory pathways that react to HCV pathogen-associated molecular patterns (PAMPs) to induce IFNs, i.e., the mitochondrial antiviral signaling protein (MAVS) and the Toll-IL-1 receptor-domain-containing adaptor-inducing IFN-β (TRIF).⁴¹ This could explain the faster second phase declines observed with the HCV protease inhibitor telaprevir³⁴ than with HCV polymerase inhibitor sofosbuvir (either alone or in combination with ribavirin or another nucleotide analog (Guedj, et al. Antivir Ther 2014; 19:211-20).

The small number of individuals in each group limits a comparison of different direct-acting antiviral regimens, and a direct comparison might be warranted in future studies. Furthermore, the patients were Chinese with genotype 1b HCV infection and without cirrhosis, of whom up to 85% had the IL28B CC genotype. IL28B CC genotype and genotype 1b HCV infection are predictive factors for an SVR, and some might argue that dual direct-acting antiviral therapies could achieve a good response in this population. Treatment with direct-acting antiviral drugs has been reported to increase the risk of recurrence of hepatocellular carcinoma in patients with previous HCV-related hepatocellular carcinoma (Reig, et al., J Hepatol Apr. 12, 2016). However, to our knowledge, this study is the first to report that SVR12 can be achieved after 3 weeks of direct-acting antiviral therapies. Normally, interferon-based treatment can achieve SVR at 24 weeks in 44-79% of patients with HCV genotype 1 infection (Nguyen and Nguyen, Aliment Pharmacol Ther 2013; 37: 921-36), but still needs more than 48 weeks of treatment. Limited data support the use of dual direct-acting antiviral therapies to shorten treatment duration. Since nucleoside analogue inhibitors do not generate second-phase declines as fast as HCV protease inhibitors, the addition of a protease inhibitor to sofosbuvir and ledipasvir or daclatasvir could allow for a shorter treatment duration, which is supported by the data presented herein.

In this study, a high proportion of patients were young women with a low baseline HCV viral load. While not wishing to be bound to a particular theory, these characteristics may be associated with a favorable response to short duration pan-oral direct-acting antiviral therapy to less than 6 weeks. Nevertheless, it has been estimated that there are at least 4 million people with such characteristics in China (Wei et al. Hepatology 2011; 54: 563A-64), and we believe these findings could benefit more patients with chronic hepatitis C worldwide in the same setting. This response-guided treatment approach can be applied, with modifications as appropriate depending on the individual, as can be readily determined by a treating physician, to patients with different ethnic backgrounds, with different genotypes, and with cirrhosis.

The instant study included a relatively small number of subjects. However, it does tend to show that one can reduce the duration of HCV treatment using on-treatment response.

The subjects were Chinese, non-cirrhotic and infected with HCV genotype 1b. They are not representative of the entire HCV infected population. However, on information and belief, the results can be reasonably extrapolated to other races/ethnicities with different genotypes and cirrhosis.

In conclusion, based on the high SVR12 rate in this study, it is possible to provide reasonable cure rates with relatively shorter durations of therapy than are commonly used with conventional therapy (Sovaldi or Harvoni). This can markedly reduce the cost of DAA regimens and make curative treatment regimens more accessible and affordable. Additionally, the combination therapy described herein can reduce the emergence of resistance and side effects. The combination therapy can also help to curtail the massive use of counterfeit medicine, which could be potentially harmful.

Additional pharmacokinetic and immunological data can be obtained by investigating the use of DAAs with high potency and non-overlapping resistance profiles tailored to specific characteristics of the subjects and viruses, to significantly shorten treatment duration without compromising efficacy.

Modeling Viral Dynamics in Subjects from the SODAPI Study

Data and 2 Sets of Analyses

Viral load data from 26 subjects who were treated with DAA therapies for 3-12 weeks was used. These 26 subjects were treated with SOF+LDV+ASV (n=12), SOF+DCV+SMV (n=6) and SOF+DCV+ASV (n=8). Subjects treated with triple therapy who had a viral load <500 IU/ml by day 2 (n=18) were treated for 3 weeks. Subjects who failed to achieve plasma HCV RNA <500 IU/mL by day 2 were switched at day 3 from triple DAAs to SOF and LDV for a total duration of 8 or 12 weeks

A multiscale model for HCV dynamics developed recently (Guedj et al., 2013), and a non-linear mixed effects modeling approach to fit data from multiple subjects simultaneously, were used. Parameter estimation was performed for 2 sets of data. The first set of data included the subset of 18 subjects who were treated with triple therapy for 3 weeks. The second set included all 26 subjects.

Model and Analytical Methods

The following analytical approximation to the solution of a multiscale HCV model derived by Guedj et al. (2013) was used:

$$V ( t ) = {V}_{0} ( {e}^{- c ( t - {t}_{0} )} + ( 1 - {ɛ}_{s} ) \frac{c ρ}{N} ( \frac{A}{B δ ( δ - c )} ( {e}^{- c ( t - {t}_{0} )} - {e}^{- δ ( t - {t}_{0} )} ) + \frac{1}{B + δ - c} ( \frac{N}{ρ} - \frac{A}{B δ} ) ( {e}^{- c ( t - {t}_{0} )} - {e}^{- ( B + δ ) ( t - {t}_{0} )} ) ) ) \\ where N = \frac{ρ ( α + S )}{δ ( ρ + μ + δ )} , A = α ( 1 - {ɛ}_{α} ) , B = ρ ( 1 - {ɛ}_{S} ) + κμ . \qquad ( 1 )$$

The symbols used in the equation are described in Table 7 below.

TABLE 5
Description and values of the symbols used in Eqn. 1.

| Symbols | Description | Value | Reference |
|---|---|---|---|
| V(t) | Viral load over time | Calculated | |
| | | from Eqn. 1 | |
| V₀ | Initial viral load | Fitted | |
| | before treatment | | |
| α | Rate constant for HCV | 40 day⁻¹ | (Guedj et al., |
| | RNA production | | 2013) |
| ρ | Export rate of HCV RNA | 8.18 day⁻¹ | (Guedj et al., |
| | | | 2013) |
| μ | Degradation rate of | 1 day⁻¹ | (Guedj et al., |
| | intracellular HCV RNA | | 2013) |
| κ | Effectiveness of DAAs in | Fitted | |
| | enhancing the degradation | | |
| | rate | | |
| c | Viral clearance rate | Fitted | |
| δ | Death rate of infected | 0.14 day⁻¹ | (Neumann et |
| | cells | | al., 1998) |
| t₀ | Pharmacological delay | Fitted | |
| ε_{α} | Inhibition of HCV RNA | Fitted | |
| | synthesis | | |
| εₛ | Inhibition of HCV RNA | Fitted | |
| | export | | |

A nonlinear mixed effects modeling approach was used, where the model was fitted to data from multiple subjects simultaneously, assuming the parameters follow a given distribution in the subject population. An advantage of this approach is that it is able to effectively deal with missing data so that those individuals with less dense sampling in our study could be included in the fitting. Using this nonlinear mixed effect modeling framework, an evaluation of whether parameter values were significantly different between groups who were treated with different DAA combinations was performed. Parameter estimation was performed using the algorithm implemented in Monolix 4.3.0 (www.lixoft.com). The data points that are below limit of detection were categorized as censored data in Monolix.

Results—18 Subjects (Who were Treated for 3 Weeks)

The multiscale model (Eqn. 1) was fitted to data from the 18 subjects who were treated for 3 weeks. In the fitting, it was assumed that the parameters V₀ and to follow a log-normal distribution among subjects, the parameter c follows a normal distribution, and that the values of log₁₀(1−ε_{α}) and log₁₀(1−εₛ) are normally distributed. It was further assumed that the parameter c does not have random effects, i.e., its value is the same for all subjects. The estimated population parameters, individual parameters and fitting results are shown below in Tables 5 and 6 and FIGS. 2A-2C. For the 18 subjects, the estimated mean efficacy of the treatments in inhibiting HCV RNA synthesis and virion secretion/export are ε_{α}=0.9962, εₛ=0.9987, respectively. The mean estimated log₁₀ baseline viral load was 5.93 (log₁₀ IU/ml). The mean estimated rate of viral clearance, c, was 29.7 day⁻¹. The mean effectiveness of the DAAs in enhancing intracellular HCV RNA degradation, κ, was 1.49, consistent with previous estimates (Guedj et al., 2013). We also tested whether parameter values were distributed significantly differently between subjects who were treated with different combinations (except for the parameter ε_{α}, because this parameter cannot be reliably estimated for individuals treated with SOF+LDV+ASV). We found no significant differences in parameter distributions. We also tested if any of the estimated parameters are significantly associated with the body mass index. There was no significant association either.

Table 6 shows the estimated population parameter values from the 18 subjects who were treated for 3 weeks derived using non-linear mixed effect model fitting.

TABLE 6

| | | Estimated standard |
|---|---|---|
| | | deviation for |
| | Estimated | random effects |
| Parameter | mean | parameters |
| log₁₀ V₀ (IU/ml) | 5.93 | 0.72 |
| −log₁₀(1 − ε_{α}) | 2.43* | 0.54 |
| −log₁₀(1 − εₛ) | 2.89* | 0.49 |
| κ | 1.49 | — |
| c (/day) | 29.7 | 8.15 |
| t₀ (day) | 0.02 | 1.1 (deviation |
| | | on a log scale) |

*The corresponding values for ε_{α} and εₛ are 0.9962 and 0.9987, respectively

Table 7 shows the estimated individual parameters from the 18 subjects who were treated for 3 weeks derived using non-linear mixed effect model fitting.

TABLE 7

| | | Log₁₀ V₀ | | | | t_{θ} | c |
|---|---|---|---|---|---|---|---|
| ID | Treatment | (copies/ml) | ε_{α} | εₛ | κ | (day) | (/day) |
| 2143 | SOF + LDV + ASV | 6.40 | 0.9987 | 0.9991 | 1.49 | 0.05 | 21.8 |
| 2153 | | 6.13 | 0.9986 | 0.9985 | 1.49 | 0.06 | 28.2 |
| 2154 | | 6.64 | 0.9991 | 0.9991 | 1.49 | 0.05 | 25.5 |
| 2162 | | 5.11 | 0.9972 | 0.9933 | 1.49 | 0.00 | 49.9 |
| 2205 | | 6.48 | 0.9993 | 0.9954 | 1.49 | 0.07 | 22.4 |
| 2207 | | 5.86 | 0.9973 | 0.9998 | 1.49 | 0.01 | 36.2 |
| Mean | | 6.10 | 0.9984 | 0.9976 | 1.49 | 0.04 | 30.7 |
| Standard Dev. | | 0.56 | 0.0009 | 0.0026 | | 0.03 | 10.8 |
| 2238 | SOF + DCV + SMV | 5.81 | 0.9874 | 0.9981 | 1.49 | 0.05 | 27.0 |
| 2279 | | 5.67 | 0.9898 | 0.9983 | 1.49 | 0.02 | 33.4 |
| 2234 | | 5.10 | 0.9965 | 0.9997 | 1.49 | 0.01 | 35.4 |
| 2315 | | 6.72 | 0.9965 | 0.9994 | 1.49 | 0.02 | 30.6 |
| 2404 | | 4.30 | 0.9965 | 0.9981 | 1.49 | 0.02 | 25.5 |
| 2405 | | 6.68 | 0.9974 | 0.9994 | 1.49 | 0.05 | 27.5 |
| Mean | | 5.71 | 0.9940 | 0.9988 | 1.49 | 0.03 | 29.9 |
| Standard Dev. | | 0.93 | 0.0043 | 0.0007 | | 0.02 | 3.9 |
| 2311 | SOF + DCV + ASV | 6.51 | 0.9977 | 0.9995 | 1.49 | 0.04 | 24.7 |
| 2326 | | 5.26 | 0.9913 | 0.9945 | 1.49 | 0.01 | 32.7 |
| 2340 | | 5.62 | 0.9870 | 0.9971 | 1.49 | 0.03 | 27.1 |
| 2341 | | 5.19 | 0.9724 | 0.9969 | 1.49 | 0.02 | 28.9 |
| 2342 | | 6.37 | 0.9974 | 0.9989 | 1.49 | 0.02 | 29.6 |
| 2387 | | 6.84 | 0.9941 | 0.9990 | 1.49 | 0.04 | 27.3 |

| | | | | | | |
|---|---|---|---|---|---|---|
| Mean | 5.96 | 0.9900 | 0.9976 | 1.49 | 0.03 | 28.4 |
| Standard Dev. | 0.70 | 0.0095 | 0.0019 | | 0.01 | 2.7 |

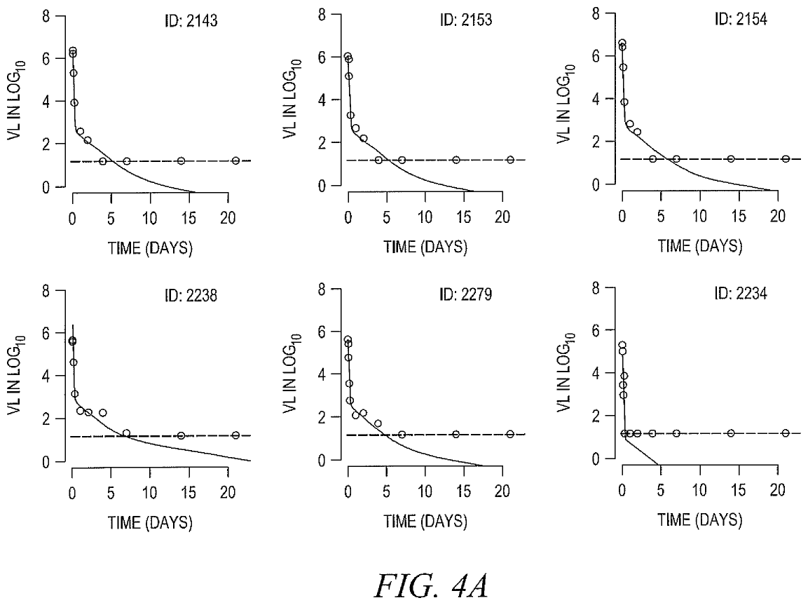

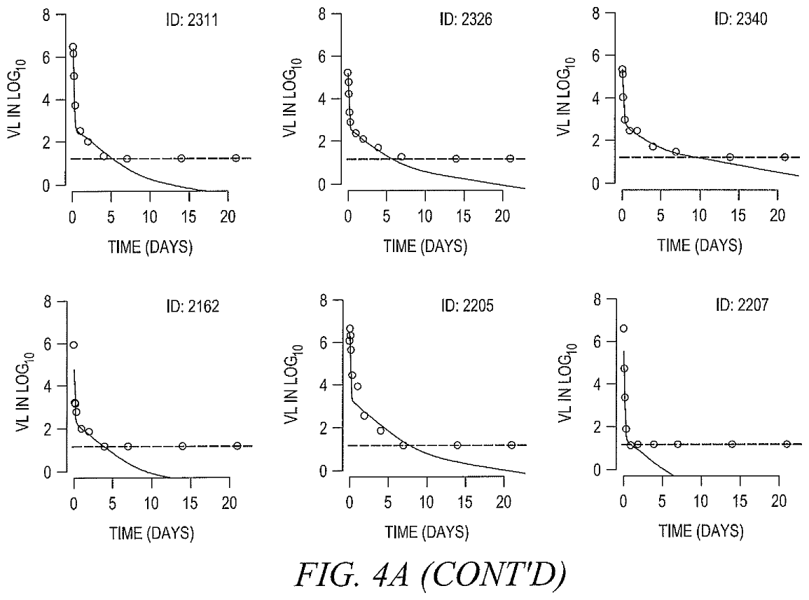

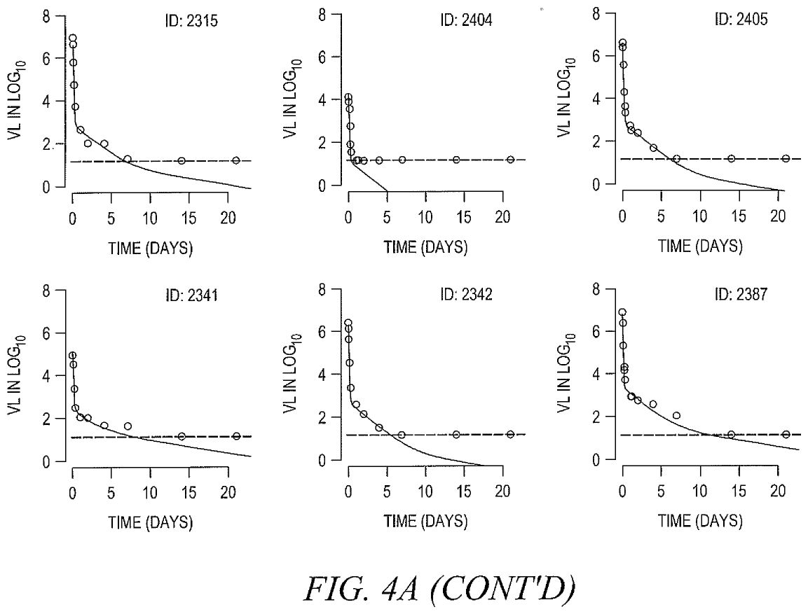

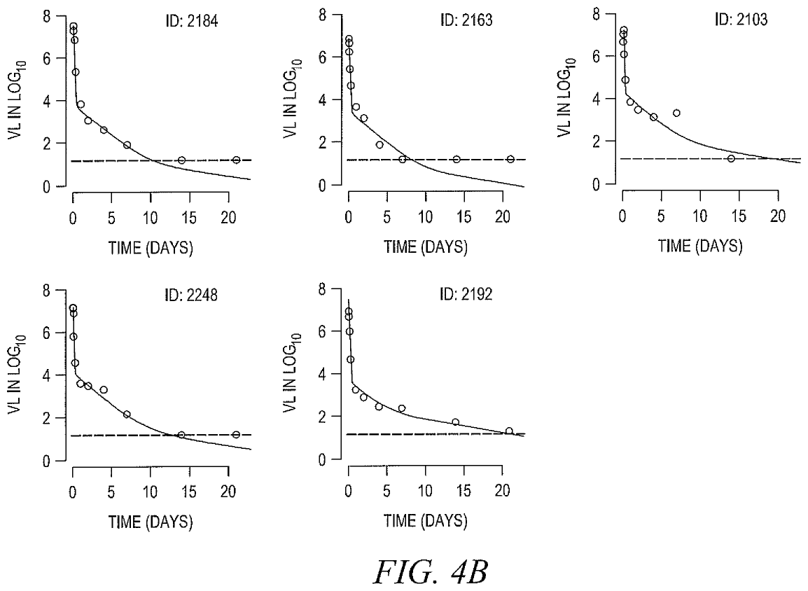

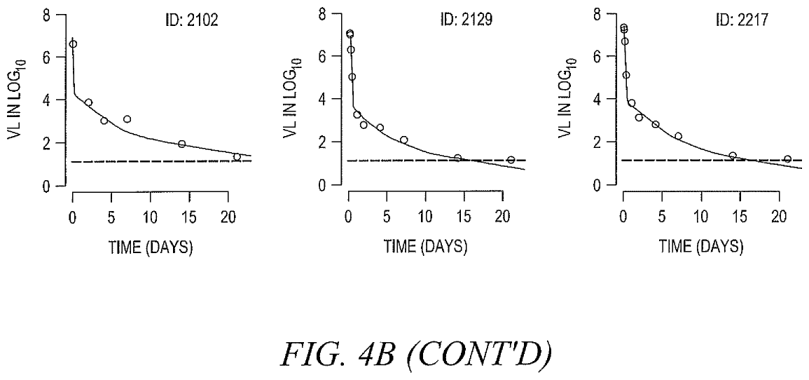

FIGS. 2A-C are charts showing the fit of the multiscale model to the data from the 18 subjects who were treated for 3 weeks. FIGS. 4A and 4B are charts showing the fit of the multiscale model to the data from the 26 patients. Quantifiable HCV RNA loads are shown as black dots, whereas viral loads below the limit of quantification (dashed lines) are shown as red dots. Simulations using the best-fit individual parameters estimated from the population fitting of the non-linear mixed effects model are shown as solid lines.

The number of subjects that did not achieve HCV RNA <500 IU/mL at 48 hours was 6 in Group I, 0 in Group 2, and 2 in Group 3.

Symbols show the median log₁₀ viral load for each treatment group, whereas the black solid line shows the mean model predicted trajectory calculated by simulating Eq. (1) 1000 times using parameters chosen randomly from the parameter distributions derived from the mixed effect modeling. The grey band shows the intraquartile range of simulation results. The dashed horizontal line indicates the assay lower limit of quantification. There appears to be three phases in the viral decline in all three treatment groups. The trend was more apparent in treatment group SOF+DCV+SMV (group 2). No significant difference was observed in viral decline rate between the three treatment groups. Significance was tested by the Kruskal-Wallis test and corrected by Dunn's test for multiple comparisons between groups. HCV denotes hepatitis C virus, SOF sofosbuvir, DCV daclatasvir, LDV ledipasvir, SMV simeprevir, and ASV asunaprevir.

Results—26 Subjects

We then fit the multiscale model (Eqn. 1) to data from all 26 subjects. The random effects parameters are assumed to follow the same type of distribution as assumed above. Using the non-linear effect modeling approach, we first tested whether parameter values are distributed significantly differently between subjects who are treated with different combinations. With the additional 6 subjects treated with SOF+LDV+ASV for the first 2 days, we were able to estimate the distribution of the parameter ε_{α} for subjects treated with this combination, thus allowing testing the differences in the distribution of ε_{α} for subjects treated with different combinations. We found no significant differences in parameter distributions.

Second, we tested if parameters are distributed differently between the 18 subjects treated for 3 weeks (‘short’ treatment) and those 8 subjects treated for more than 3 weeks with Harvoni (‘long’ treatment) by setting the treatment duration as a categorical covariate. We found that parameter V₀ was distributed significantly differently between subjects who were treated for 3 weeks and subjects who were treated for more than 3 weeks after Bonferroni correction for multiple tests (see Tables 9 and 10 and FIG. 3 for the estimated population parameters, individual parameters and fitting results). Subjects who were treated for 3 weeks had a lower estimated baseline viral load than subjects treated for more than 3 weeks (population means of log₁₀ V₀ are 5.81 and 7.23, respectively, p-value: 1.5×10⁻⁶). The estimated mean efficacy of the treatments in inhibiting HCV RNA synthesis and virion secretion/export are ε_{α}=0.9983, εₛ=0.9993, respectively, similar to the results above using data from 18 subjects. The mean effectiveness of the DAAs in affecting intracellular HCV RNA degradation, κ, was 0.68. The estimated mean viral clearance rate and the estimated mean pharmacological delay were 23.9 day⁻¹ and 0.5 hr, respectively, consistent with previous estimates (Guedj et al., 2013).

Table 8 shows estimated population parameter values from the 26 subjects who were treated with DAA combinations derived using non-linear mixed effect model fitting

TABLE 8

| | | Estimated standard |
|---|---|---|
| | | deviation for |
| | Estimated | random effects |
| Parameter | mean | parameters |
| log₁₀ V_{0, short} (IU/ml) | 5.81* | 0.66 |
| log₁₀ V_{0, long} (IU/ml) | 7.23* | |
| −log₁₀(1 − ε_{α}) | 2.77** | 0.50 |
| −log₁₀(1 − εₛ) | 3.18** | 0.45 |
| κ | 0.68 | — |
| c (/day) | 23.1 | 0.32 |
| t₀ (day) | 0.03 | 0.87 (deviation |
| | | on a log scale) |

We assumed that parameter V₀ was distributed differently between subjects who were treated for 3 weeks (‘short’ duration) and persons who were treated for more than 3 weeks (‘long’ duration).
*The p-value for the differences between the distributions of log₁₀ V_{0, short} and log₁₀ V_{0, long} is 1.5 × 10⁻⁶.
**The corresponding values for ε_{α} and εₛ are 0.9983 and 0.9993, respectively.

Table 9 shows estimated individual parameters from the 26 subjects who were treated with DAA combinations, derived using non-linear mixed effect model fitting.

TABLE 9

| | | | Log₁₀ V₀ | | | | t_{θ} | c |
|---|---|---|---|---|---|---|---|---|
| Groups | ID | Treatment | (copies/ml) | ε_{α} | εₛ | κ | (day) | (/day) |
| 3 weeks | 2143 | SOF + LDV + ASV | 6.39 | 0.9990 | 0.9997 | 0.68 | 0.05 | 22.1 |
| treatment | 2153 | | 6.12 | 0.9990 | 0.9995 | 0.68 | 0.05 | 22.8 |
| | 2154 | | 6.52 | 0.9992 | 0.9997 | 0.68 | 0.05 | 22.5 |
| | 2162 | | 4.81 | 0.9987 | 0.9961 | 0.68 | 0.01 | 24.9 |
| | 2205 | | 6.44 | 0.9993 | 0.9990 | 0.68 | 0.08 | 21.6 |
| | 2207 | | 5.49 | 0.9985 | 0.9999 | 0.68 | 0.01 | 25.8 |
| | 2238 | SOF + DCV + SMV | 5.87 | 0.9964 | 0.9990 | 0.68 | 0.04 | 23.0 |
| | 2279 | SOF + DCV + ASV | 5.53 | 0.9981 | 0.9988 | 0.68 | 0.02 | 24.5 |
| | 2234 | | 4.81 | 0.9985 | 0.9998 | 0.68 | 0.02 | 25.9 |
| | 2315 | | 6.59 | 0.9982 | 0.9997 | 0.68 | 0.02 | 23.9 |
| | 2404 | | 4.29 | 0.9985 | 0.9991 | 0.68 | 0.02 | 22.9 |
| | 2405 | | 6.57 | 0.9991 | 0.9997 | 0.68 | 0.04 | 23.4 |
| | 2311 | | 6.38 | 0.9989 | 0.9998 | 0.68 | 0.04 | 22.4 |
| | 2326 | | 5.16 | 0.9964 | 0.9966 | 0.68 | 0.02 | 23.8 |
| | 2340 | | 5.57 | 0.9931 | 0.9982 | 0.68 | 0.03 | 23.1 |
| | 2341 | | 5.13 | 0.9900 | 0.9973 | 0.68 | 0.02 | 23.7 |
| | 2342 | | 6.19 | 0.9989 | 0.9995 | 0.68 | 0.03 | 23.2 |
| | 2387 | | 6.76 | 0.9975 | 0.9994 | 0.68 | 0.03 | 23.0 |
| | Mean | | 5.81 | 0.9976 | 0.9989 | 0.68 | 0.03 | 23.5 |
| | Standard Dev. | | 0.74 | 0.0024 | 0.0011 | | 0.02 | 1.2 |
| >3 weeks | 2184 | SOF + LDV + ASV | 7.43 | 0.9993 | 0.9997 | 0.68 | 0.08 | 21.3 |
| treatment | 2163 | then SOF + LDV | 6.86 | 0.9995 | 0.9995 | 0.68 | 0.03 | 21.8 |
| | 2103 | | 7.18 | 0.9978 | 0.9989 | 0.68 | 0.06 | 22.3 |
| | 2102 | | 6.95 | 0.9975 | 0.9974 | 0.68 | 0.04 | 22.9 |
| | 2129 | | 7.18 | 0.9980 | 0.9996 | 0.68 | 0.06 | 21.2 |
| | 2217 | SOF + DCV + ASV | 7.46 | 0.9989 | 0.9996 | 0.68 | 0.06 | 21.5 |
| | 2248 | then SOF + LDV | 7.30 | 0.9993 | 0.9993 | 0.68 | 0.03 | 22.7 |
| | 2192 | | 7.53 | 0.9966 | 0.9998 | 0.68 | 0.04 | 23.7 |

| | | | | | | |
|---|---|---|---|---|---|---|
| Mean | 7.24 | 0.9984 | 0.9992 | 0.68 | 0.05 | 22.2 |
| Standard Dev. | 0.24 | 0.0011 | 0.0008 | | 0.02 | 0.9 |

We assumed that parameter V₀ is distributed differently between subjects who were treated for 3 weeks and persons who were treated for more than 3 weeks.

FIGS. 2A-C show a fit of the multiscale model to data from the 26 subjects who were treated with DAAs combinations assuming V₀ was distributed differently between subjects who were treated for 3 weeks and subjects who were treated for more than 3 weeks. Quantifiable HCV RNA loads are shown as black dots, whereas viral loads below the limit of quantification (dashed lines) are shown as red dots. Simulations using best-fit individual parameters estimated from the population fitting of the non-linear mixed effects model are shown as solid lines.

Table 10 Shows Baseline HCV NS3/4A, NS5A, and NS5B Resistance-Associated Variants (RAs) for 26 Subjects.

TABLE 10

| | | | | | HCV RNA > 500 | |
|---|---|---|---|---|---|---|
| | | | | | IU/mL by | |
| | | | | | day 2 >3 | |
| | | HCV RNA < 500 IU/mL by day 2 | | | weeks | |
| | | 3 weeks treatment | | | treatment | |
| | | SOF + LDV + ASV | SOF + DSV + SMV | SOF + DSV + ASV | SOF + | — |
| — | | (Group 1) | (Group 2) | (Group 3) | LDV | P- |
| DAA-RAVs | | n = 6 | n = 6 | n = 6 | n = 8 | value |
| NS3/4A | I132V | 4 (66.7) | 5 (80.0) | 4 (66.7) | 5 (62.5) | >0.05 |
| N (%) | I132V, | 1 (16.7) | — | — | — | |
| | T54S | | | | | |
| | I132V, | — | — | 1 (16.7) | — | |
| | V36L | | | | | |
| | I132V, | — | 1 (20.0) | — | — | |
| | S122R | | | | | |
| | I32V, | — | — | — | 2 (25.0) | |
| | D168H/N/Y | | | | | |
| | None | 1 (16.7) | — | 1 (16.7) | 1 (12.5) | |
| NS5A | Q30R | 4 (66.7) | 6 (100) | 5 (83.3) | 3 (37.5) | >0.05 |
| N (%) | Q30R, | 1 (16.7) | — | — | — | |
| | H54N | | | | | |
| | Q30R, | — | — | — | 2 (25.0) | |
| | Y93H | | | | | |
| | Q30R, | — | — | — | 1 (12.5) | |
| | L31 F | | | | | |
| | Q30R, | 1 (16.7) | — | 1 (16.6) | — | |
| | L31F, | | | | | |
| | Y93H | | | | | |
| | None | — | — | — | 2 (25.0) | |
| NS5B | R422K | — | — | — | 1 (12.5) | — |
| N (%) | None | 6 (100) | 6 (100) | 6 (100) | 7 (87.5) | |

The NS5B region was partially amplified using primer sequences as follows (5′-3′ orientation): 1-fwd: GGCGGAATTCCTGGTCATAGCCTCCGTGAA, and 2-rev TGGGGATCCCGTATGATACCCGCTGCTTTGA. After purification using QIAquick PCR purification kit (Qiagen) or ExoSAP It kit (Affymetrix, Santa Clara, Calif., USA), primer extension sequencing was performed by GENEWIZ, Inc (South Plainfield, N.J.) using Applied Biosystems BigDye version 3.1. The reactions were then performed on an Applied Biosystem's 3730xl DNA Analyzer.

One (4.2%) NSSB RAV (R422K) associated with resistance to filibuvir or lomibuvir (VX222) was detected. Interestingly, we did not find the baseline polymorphism L320F, which in combination with another mutation (L159F) could confer resistance to NS5B inhibitors (Table S6). Three of the eight subjects without RVR and two of the 18 with RVR had NS5A RAVs, either Y93H (n=2) or L31F (n=1) without RVR, and either H54N (n=1) or L3 IV plus Y93H (n=1) with RVR (p=0.08).

Table 11 shows plasma concentrations of anti-HCV drugs in subjects treated with DAA combinations (ng/mL)

TABLE 11

| | | | Time | | | | | |
|---|---|---|---|---|---|---|---|---|
| Groups | ID | Treatment | (h) | SOF | LDV | DCV | ASV | SMV |
| 3 weeks | 2143 | SOF + LDV + ASV | 8 | 4.54 | 47.3 | — | 6.78 | — |
| treatment | | | 24 | 1.17 | 23.7 | — | 1.34 | — |
| | | | 48 | 0.92 | 48.7 | — | — | — |
| | 2153 | | 8 | 10.4 | 55.5 | — | 50.7 | — |
| | | | 24 | 0.99 | 22.2 | — | 86.2 | — |
| | | | 48 | 0.81 | 44.8 | — | 48.7 | — |
| | 2154 | | 8 | 7.17 | 202 | — | 215 | — |
| | | | 24 | 0.94 | 49.8 | — | 10.4 | — |
| | | | 48 | 4.76 | 99.2 | — | 15.0 | — |
| | 2162 | | 8 | 5.52 | 112 | — | 144 | — |
| | | | 24 | 8.92 | 254 | — | 25.1 | — |
| | | | 48 | 6.35 | 427 | — | 701 | — |
| | 2205 | | 8 | 4.22 | 131 | — | 14.5 | — |
| | | | 24 | 1.23 | 32.6 | — | 157 | — |
| | | | 48 | 0.64 | 61.5 | — | 220 | — |
| | 2207 | | 8 | 18.2 | 469 | — | 302 | — |
| | | | 24 | 2.13 | 466 | — | 626 | — |
| | | | 48 | 1.59 | 153 | — | 224 | — |
| | 2238 | SOF + DCV + SMV | 8 | 2.35 | — | 316 | — | 693 |
| | 2279 | | 8 | 0.26 | — | 573 | — | 528 |
| | 2234 | | 8 | 14.7 | — | 1451 | — | 139 |
| | | | 24 | 2412 | — | 2569 | — | 48.0 |
| | | | 48 | 3174 | — | 3045 | — | 95.7 |
| | 2315 | | 8 | 0.42 | — | 500 | — | 585 |
| | | | 24 | — | — | 101 | — | 83.7 |
| | | | 48 | 1796 | — | 786 | — | 149 |
| | 2404 | | 8 | — | — | 641 | — | 396 |
| | | | 24 | 2114 | — | 1481 | — | 63.9 |
| | | | 48 | 1549 | — | 1587 | — | 131 |
| | 2405 | | 8 | 1.45 | — | 358 | — | 173 |
| | | | 24 | 1923 | — | 600 | — | 94.8 |
| | | | 48 | 1227 | — | 683 | — | 30.6 |
| | 2311 | SOF + DCV + ASV | 8 | 0.55 | — | 470 | 7.15 | — |
| | | | 24 | 1.09 | — | 249 | 22.7 | — |
| | | | 48 | 1.41 | — | 254 | 17.7 | — |
| | 2326 | | 8 | — | — | 816 | 26.5 | — |
| | | | 24 | 0.28 | — | 193 | 13.3 | — |
| | | | 48 | 0.80 | — | 243 | 19.0 | — |
| | 2340 | | 8 | 0.28 | — | 627 | 11.0 | — |
| | | | 24 | 0.27 | — | 127 | 9.19 | — |
| | | | 48 | 7.55 | — | 126 | 6.01 | — |
| | 2341 | | 8 | 2.03 | — | 739 | 18.8 | — |
| | | | 24 | — | — | 170 | 20.8 | — |
| | | | 48 | 292 | — | 1632 | 154 | — |
| | 2342 | | 8 | 13.5 | — | 594 | 24.9 | — |
| | | | 24 | 192 | — | 172 | 19.3 | — |
| | | | 48 | 12.7 | — | 232 | 9.84 | — |
| | 2387 | | 8 | 0.47 | — | 302 | 9.34 | — |
| | | | 24 | 256 | — | 115 | 78.1 | — |
| | | | 48 | 1807 | — | 1460 | 344 | — |
| >3 weeks | 2184 | SOF + LDV + ASV | 24 | — | 7.70 | — | 24.1 | — |
| treatment | | Then SOF + LDV | 48 | — | 45.9 | — | 54.2 | — |
| | 2163 | | 8 | — | 112 | — | 19.0 | — |
| | | | 24 | 202 | 388 | — | 200 | — |
| | | | 48 | — | — | — | — | — |
| | 2103 | | 8 | — | 244 | — | 14.0 | — |
| | | | 24 | 1591 | 72.2 | — | 237 | — |
| | 2129 | | 8 | 0.67 | 272 | — | 190 | — |
| | | | 24 | 0.61 | 50.9 | — | 10.1 | — |
| | | | 48 | — | 60.1 | — | 9.90 | — |
| | 2217 | | 8 | 0.58 | 192 | — | 55.1 | — |
| | | | 24 | — | 53.0 | — | 29.7 | — |
| | | | 48 | — | 89.3 | — | 25.6 | — |
| | 2248 | SOF + DCV + ASV | 8 | — | — | 680 | 20.6 | — |
| | | Then SOF + LDV | 24 | 11.2 | — | 237 | 22.0 | — |
| | | | 48 | 631 | — | 143 | 1.30 | — |
| | 2192 | | 8 | 9.99 | — | 1203 | 40.7 | — |
| | | | 24 | — | — | 249 | 93.9 | — |
| | | | 48 | — | — | 378 | 10.3 | — |

| | | | | | | |
|---|---|---|---|---|---|---|
| Limit of Quantification (LOQ) | — | 0.26 | 3.56 | 0.74 | 1.20 | 0.75 |

— indicates below LOQ

Fifty μL plasma was extracted with 3 mL of ethyl acetate containing abacavir (as internal standard). The supernatant was collected, air-dried then reconstituted in 2 mM ammonium acetate solution with 75% methanol. Five target compounds were simultaneously monitored and quantified by LC-MS/MS in the multiple reaction monitoring (MRM) mode, including sofosbuvir (m/z 530.2a243.2), daclatasvir (m/z 739.5à339.3), ledipasvir (m/z 889.4à637.3), asunaprevir (m/z 748.4à648.4) and simeprevir (m/z 750.4à315.2). SOF concentrations were measured with a wide range from BLOQ to 3,174 ng/mL; LDV and DCV were in the range of BLOQ to 469 ng/mL and 101 to 3045 ng/mL; ASV and SMV were in the range of BLOQ to 626 ng/mL and 30.6 to 693 ng/mL, respectively. These results indicated the drug levels in subjects at 8, 24 and 48 hr after first treatment.

Supplementary Documents

Additional experimental data is described below. Table 12 shows demographic and clinical characteristics of the fast responders and slow responders at baseline*

TABLE 12

| | Fast responders | Slow responders | |
|---|---|---|---|
| | (plasma HCV RNA < 500 | (plasma HCV ≥ RNA 500 | |
| | IU/mL by Day 2) | IU/mL by Day 2) | P |
| | n = 18 | n = 8 | value |
| Age -- yr | | | |
| Mean | 37 | 36 | 0.84 |
| Range | 21-66 | 22-50 | |
| Male sex -- no. (%) | 6 (33.3) | 4 (50.0) | 0.35 |
| Body-mass index (kg/m²)^{#} | | | |
| Mean | 21.4 | 23.7 | 0.07 |
| Range | 15.9-32.1 | 18.5-28.3 | |
| HCV RNA -- Log10 IU/mL | 6.0 ± 0.8 | 7.0 ± 0.3 | <0.001 |
| HCV RNA ≥ 800,000 IU/mL -- | 10 (55.5) | 8 (100) | 0.03 |
| no. (%) | | | |
| Previous PR treatment - no. (%) | | | 0.17 |
| Experienced | 6 (33.3) | 5 (62.5) | |
| Naïve | 12 (66.7) | 3 (37.5) | |
| Previous PR response -- no. (%)^{&} | | | 0.99 |
| Relapser | 4 (66.7) | 3 (60.0) | |
| Partial responder | 1 (16.7) | 2 (40.0) | |
| Null responder | 1 (16.7) | 0 (0) | |
| IL28B genotype -- no. (%) | | | 0.67 |
| CC | 11 (61.1) | 6 (75.0) | |
| CT | 7 (38.9) | 2 (25.0) | |
| IFNL4 genotype -- no. (%) | | | 0.99 |
| TT/TT | 13 (72.2) | 6 (75.0) | |
| ΔG/TT | 5 (27.8) | 2 (25.0) | |
| Liver stiffness (kPa)^{$} | 5.8 ± 1.5 | 7.3 ± 2.5 | 0.1 |
| Fibrosis stage (METAVIR) -- no. (%) | | | 0.1 |
| F0-F1 (Fibroscan score <= 7.3) | 14 (77.8) | 5 (62.5) | |
| F2(7.3 < Fibroscan score <= 9.7) | 4 (22.2) | 1 (12.5) | |
| F3(9.7 < Fibroscan score <= 14.7) | 0 (0) | 2 (25.0) | |

*Plus-minus values are mean +/− SD. The Kruskal-Wallis test and Fisher's exact test were used to test for the difference across treatment groups for continuous and categorical variables, respectively.

Π The body-mass index (BMI) is the weight in kilograms divided by the square of the height in meters.

& Data indicate the response to the most recent PR-based regimen. Relapser is the subject who received at least 36 weeks of pegIFN/RBV for the treatment of HCV and was undetectable at the end of treatment, but HCV RNA was detectable within 52 weeks of treatment follow-up. Partial responder is the subject who received at least 20 weeks of pegIFN/RBV for the treatment of HCV and achieved ≥2 log₁₀ IU/ml reduction in HCV RNA at Week 12, but failed to achieve HCV RNA undetectable at the end of treatment. Null responder is the subject who received at least 20 weeks of pegIFN/RBV for the treatment of HCV and failed to achieve a 2 log₁₀ reduction in HCV RNA at Week 12.

$ The liver stiffness was determined by Fibroscan (Eschosens, France). No subject in this study had a cirrhosis (Fibrosis score >14.7 kPa).

Table 13 shows the proportion of individuals with HCV RNA level lower than the quantification limit*

TABLE 13

| Fast responders | | Slow responders | |
|---|---|---|---|
| (plasma HCV RNA < 500 | | (plasma HCV RNA ≥ 500 | |
| IU/mL by Day 2) | | IU/mL by Day 2) | |
| n = 18 | | n = 8 | |
| n | % (95% CI) | n | % (95% CI) |

During treatment period

| | | | | |
|---|---|---|---|---|
| Day 2 | 3 | 16.7 (5.0-43.3) | 0 | 0 |
| Day 4 | 7 | 38.9 (18.6-63.9) | 0 | 0 |
| Day 7 | 12 | 66.7 (40.9-85.2) | 1 | 12.5 (1.3-60.5) |
| Week 2 | 17 | 94.4 (65.7-99.3) | 4 | 50 (17.4-82.6) |
| Week 3 | 18 | 100 | 6 | 75 (33.2-94.8) |
| Week 4 | na | na | 8 | 100 |
| Week 8 | na | na | 8 | 100 |
| Week 12 | na | na | 8 | 100 |

During post-treatment period

| | | | | |
|---|---|---|---|---|
| At week 4 | 18 | 100 | 8 | 100 |
| At week 12 | 18 | 100 | 8 | 100 |

*The limit of quantification for plasma HCV RNA by Roche COBAS Taqman test was 25 IU/ml.
ρ = 0.003 for the difference in the median time for HCV RNA level to be lower than the quantification limit between these two groups.
Significance was tested by Wilcoxon-Mann-Whitney test.
Na = not applicable.

### REFERENCES

1. Mohd Hanafiah K, Groeger J, Flaxman A D, Wiersma S T. Global epidemiology of hepatitis C virus infection: new estimates of age-specific antibody to HCV seroprevalence. Hepatology 2013; 57:1333-42.
2. Messina J P, Humphreys I, Flaxman A, et al. Global distribution and prevalence of hepatitis C virus genotypes. Hepatology 2015; 61:77-87.
3. Liu C H, Kao J H. Nanomedicines in the treatment of hepatitis C virus infection in Asian patients: optimizing use of peginterferon alfa. Int J Nanomedicine 2014; 9:2051-67.
4. Qin Q, Smith M K, Wang L, et al. Hepatitis C virus infection in China: an emerging public health issue. J Viral Hepat 2015; 22:238-44.
5. Alter H J, Seeff L B. Recovery, persistence, and sequelae in hepatitis C virus infection: a perspective on long-term outcome. Semin Liver Dis 2000; 20:17-35.
6. Lauer G M, Walker B D. Hepatitis C virus infection. N Engl J Med 2001; 345:41-52.
7. AASLD-IDSA HCV Guidance Panel. Hepatitis C guidance: AASLD-IDSA recommendations for testing, managing, and treating adults infected with hepatitis C virus. Hepatology 2015; 62:932-54.
8. European Association for Study of Liver. EASL Recommendations on Treatment of Hepatitis C 2015. J Hepatol 2015; 63:199-236.
9. Afdhal N, Zeuzem S, Kwo P, et al. Ledipasvir and sofosbuvir for untreated HCV genotype 1 infection. N Engl J Med 2014; 370:1889-98.
10. Afdhal N, Reddy K R, Nelson D R, et al. Ledipasvir and sofosbuvir for previously treated HCV genotype 1 infection. N Engl J Med 2014; 370:1483-93.
11. Kowdley K V, Gordon S C, Reddy K R, et al. Ledipasvir and sofosbuvir for 8 or 12 weeks for chronic HCV without cirrhosis. N Engl J Med 2014; 370:1879-88.
12. Sulkowski M S, Gardiner D F, Rodriguez-Torres M, et al. Daclatasvir plus sofosbuvir for previously treated or untreated chronic HCV infection. N Engl J Med 2014; 370:211-21.
13. Jayasekera C R, Barry M, Roberts L R, Nguyen M H. Treating hepatitis C in lower-income countries. N Engl J Med 2014; 370:1869-71.
14. McCarthy M. Fake medicines are undermining global efforts to combat infectious disease, says US journal. BMJ 2015; 350:h2137.
15. Attaran A. Stopping murder by medicine: introducing the Model Law on Medicine Crime. Am J Trop Med Hyg 2015; 92:127-32.
16. Hagan L M, Sulkowski M S, Schinazi R E. Cost analysis of sofosbuvir/ribavirin versus sofosbuvir/simeprevir for genotype 1 hepatitis C virus in interferon-ineligible/intolerant individuals. Hepatology 2014; 60:37-45.
17. Hill A, Khoo S, Fortunak J, Simmons B, Ford N. Minimum costs for producing hepatitis C direct-acting antivirals for use in large-scale treatment access programs in developing countries. Clin Infect Dis 2014; 58:928-36.
18. Foster G R. Shorter treatments for hepatitis C: another step forward? Lancet 2015; 385:1054-5.
19. Gane E J, Stedman C A, Hyland R H, et al. Efficacy of nucleotide polymerase inhibitor sofosbuvir plus the NS5A inhibitor ledipasvir or the NS5B non-nucleoside inhibitor GS-9669 against HCV genotype 1 infection. Gastroenterology 2014; 146:736-43 el.
20. Kohli A, Osinusi A, Sims Z, et al. Virological response after 6 week triple-drug regimens for hepatitis C: a proof-of-concept phase 2A cohort study. Lancet 2015; 385:1107-13.
21. Perelson A S, Guedj J. Modelling hepatitis C therapy-predicting effects of treatment. Nature reviews Gastroenterology & hepatology 2015.
22. Dixit N M, Layden-Almer J E, Layden T J, Perelson A S. Modelling how ribavirin improves interferon response rates in hepatitis C virus infection. Nature 2004; 432:922-4.
23. Snoeck E, Chanu P, Lavielle M, et al. A comprehensive hepatitis C viral kinetic model explaining cure. Clinical pharmacology and therapeutics 2010; 87:706-13.
24. Guedj J, Pang P S, Denning J, et al. Analysis of hepatitis C viral kinetics during administration of two nucleotide analogues: sofosbuvir (GS-7977) and GS-0938. Antivir Ther 2014; 19:211-20.
25. Guedj J, Dahari H, Shudo E, Smith P, Perelson A S. Hepatitis C viral kinetics with the nucleoside polymerase inhibitor mericitabine (RG7128). Hepatology 2012; 55:1030-7.
26. Guedj J, Dahari H, Rong L, et al. Modeling shows that the NS5A inhibitor daclatasvir has two modes of action and yields a shorter estimate of the hepatitis C virus half-life. Proc Natl Acad Sci USA 2013; 110:3991-6.
27. Rong L, Guedj J, Dahari H, el al. Analysis of hepatitis C virus decline during treatment with the protease inhibitor danoprevir using a multiscale model. PLoS Comp. Biol. 2013; 9, e1002959
28. McGivern, D R, Masaki T, Williford S, et al. Kinetic analyses reveal potent and early blockade of hepatitis C virus assembly by NS5A inhibitors. Gastroenterolgy 2014; 147, 453-462.
29. McGivern, D R, Masaki T, Lovellet W al. Protease inhibitors block multiple functions of the NS3/4A protease-helicase during the hepatitis C virus life cycle. J Virol 2015; 89, 5362-5370.
30. Sulkowski M, Hezode C, Gerstoft J, et al. Efficacy and safety of 8 weeks versus 12 weeks of treatment with grazoprevir (MK-5172) and elbasvir (MK-8742) with or without ribavirin in patients with hepatitis C virus genotype 1 mono-infection and HIV/hepatitis C virus co-infection (C-WORTHY): a randomised, open-label phase 2 trial. Lancet 2015; 385:1087-97.
31. Poordad F, Lawitz E, Gutierrez J A, et al. C-swift: grazoprevir/elbasvir+sofosbuvir in cirrhotic and noncirrhotic, treatment-naive patients with hepatitis C virus genotype 1 infection, for durations of 4, 6 or 8 weeks and genotype 3 infection for durations of 8 or 12 weeks. Journal of Hepatology 2015; 62:S192-S3.
32. Kattakuzhy S, Sidharthan S, Wilson E, et al. Predictors of sustained viral response to 4-6 week duration therapy with ledipasvir+sofosbuvir+GS-9451+/−G S-9669 in early and advanced fibrosis (NIH/UMD synergy trial). Journal of Hepatology 2015; 62:S669.
33. Lawitz E J, Gruener D, Hill J M, et al. A phase 1, randomized, placebo-controlled, 3-day, dose-ranging study of GS-5885, an NS5A inhibitor, in patients with genotype 1 hepatitis C. J Hepatol 2012; 57:24-31.
34. Guedj J, Perelson A S. Second-phase hepatitis C virus RNA decline during telaprevir-based therapy increases with drug effectiveness: implications for treatment duration. Hepatology 2011; 53:1801-8.
35. Svarovskaia E S, Dvory-Sobol H, Parkin N, et al. Infrequent development of resistance in genotype 1-6 hepatitis C virus-infected subjects treated with sofosbuvir in phase 2 and 3 clinical trials. Clin Infect Dis 2014; 59:1666-74.
36. Yukl S A, Boritz E, Busch M, et al. Challenges in detecting HIV persistence during potentially curative interventions: a study of the Berlin patient. PLoS Pathog 2013; 9:e1003347.
37. Saez-Cirion A, Bacchus C, Hocqueloux L, et al. Post-treatment HIV-1 controllers with a long-term virological remission after the interruption of early initiated antiretroviral therapy ANRS VISCONTI Study. PLoS Pathog 2013; 9:e1003211.
38. Conway J M, Perelson A S. Post-treatment control of HIV infection. Proc Natl Acad Sci USA 2015; 112:5467-72.
39. Lin J C, Habersetzer F, Rodriguez-Torres M, et al. Interferon gamma-induced protein 10 kinetics in treatment-naive versus treatment-experienced patients receiving interferon-free therapy for hepatitis C virus infection: implications for the innate immune response. J Infect Dis 2014; 210:1881-5.
40. Rehermann B. Pathogenesis of chronic viral hepatitis: differential roles of T cells and N K cells. Nat Med 2013; 19:859-68.
41. Horner S M, Gale M, Jr. Regulation of hepatic innate immunity by hepatitis C virus. Nat Med 2013; 19:879-88.
42. Neumann A, Lam N P, Perelson A S, et al. (1998). Hepatitis C viral dynamics in vivo and the antiviral efficacy of interferon-alpha therapy. Science 282(5386): 103-107.

It should be understood that the above-described embodiments and the examples are given by way of illustration, not limitation. Various changes and modifications within the scope of the present invention will become apparent to those skilled in the art from the present description.

All references (patent and non-patent) cited above are incorporated by reference into this patent disclosure. The discussion of those references is intended merely to summarize the assertions made by their authors. No admission is made that any reference (or a portion of any reference) is relevant prior art (or prior art at all). Applicants reserve the right to challenge the accuracy and pertinence of the cited references.

1. A formulation for treating hepatitis C viral infections comprising:
(i)
a) Sofusbuvir at a dosage of 400-1600 mg “every day”;
b) Ledipasvir at a dosage of 90-360 mg “every day” or Daclatasvir at a dosage of 60-240 mg “every day”; and
c) Simeprevir at a dosage of 150-600 mg “every day” or Asunaprevir at a dosage of 100-400 mg “twice a day”, and
(ii) a pharmaceutically-acceptable carrier or excipient.

2. The formulation of claim 1, wherein two of the three components are present in a first unit dosage form for oral administration and one of the three components is present in a second unit dosage form for oral administration.

3. The formulation of claim 1, comprising a further anti-HCV compound.

4. The formulation of claim 1, wherein the combination of anti-HCV compounds comprises Solvaldi at a dosage of 400-1600 mg “every day”, Ledipasvir at a dosage of 90-360 mg “every day”, and Simeprevir at a dosage of 150-600 mg.

5. The formulation of claim 1, wherein the combination of anti-HCV compounds comprises Sovaldi at a dosage of 400-1600 mg “every day”, Ledipasvir at a dosage of 90-360 mg “every day”, and Asunaprevir at a dosage of 100-400 mg “twice a day”.

6. The formulation of claim 1, wherein the combination of anti-HCV compounds comprises Sovaldi at a dosage of 400-1600 mg “every day”, Daclatasvir at a dosage of 60-240 mg “every day”, and Simeprevir at a dosage of 150-600 mg.

7. The formulation of claim 1, wherein the combination of anti-HCV compounds comprises Sovaldi at a dosage of 400-1600 mg “every day”, Daclatasvir at a dosage of 60-240 mg “every day”, and Asunaprevir at a dosage of 100-400 mg “twice a day”.

8. The formulation of claim 1, further comprising a JAK inhibitor.

9. The formulation of claim 9, wherein the JAK inhibitor is Ruxolitinib Baracitinib, or Tofacitinib.

10. A method for treating hepatitis C viral infections to provide a cure within four weeks of the initiation of treatment for a subset of patients, comprising:
a) administering a formulation, comprising (i)
Sofusbuvir at a dosage of 400-1600 mg “every day”;
Ledipasvir at a dosage of 90-360 mg “every day” or Daclatasvir at a dosage of 60-240 mg “every day”;
Simeprevir at a dosage of 150-600 mg “every day” or Asunaprevir at a dosage of 100-400 mg “twice a day”, and
a pharmaceutically-acceptable carrier or excipient,
to an HCV-positive patient for a period of two days,
b) measuring the number of plasma HCV RNA copies, and if the patient shows a rapid virologic response, defined as having less than 500 IU/ml plasma HCV RNA copies, continuing the therapy for up to three weeks, until the patient has less than 25 IU/ml plasma HCV RNA copies, at which point the patient is considered to have been successfully cured in four weeks or less,
and if the limit of less than 25 IU/ml plasma HCV RNA copies is not achieved within three weeks of therapy, ceasing therapy with the formulations of claim 1, and, instead, administering conventional anti-HCV therapy.

11. The method of claim 10, further comprising screening the HCV positive patient to determine one or both of the patient's HCV base viral load and whether or not the patient has cirrhosis of the liver before initiating treatment.

12. The method of claim 10, wherein the HCV is HCV of subtype 1a or 1b.

13. The method of claim 12, wherein the HCV is HCV of subtype 1b.
